(19) United States
(12) Patent Application Publication
(10) Pub. No.: US 2022/0409273 A1
(43) Pub. Date: Dec. 29, 2022

(54) CHEMICAL NEUROMODULATION AGENT DELIVERY

(71) Applicant: Paul J. Coates, Corte Madera, CA (US)

(72) Inventor: Paul J. Coates, Corte Madera, CA (US)

(21) Appl. No.: 17/808,494

(22) Filed: Jun. 23, 2022

Related U.S. Application Data
(60) Provisional application No. 63/214,479, filed on Jun. 24, 2021.

(51) Int. Cl. A61B 18/18

(52) U.S. Cl. CPC A61B 18/18; A61B 2018/00434

(57) ABSTRACT

A method may include positioning a distal portion of a neuromodulation catheter in a first renal vessel of a patient. The distal portion may include a plurality of therapeutic elements arranged around a perimeter of the distal portion. The method also may include imaging the distal portion to visualize positions of the plurality of therapeutic elements; manipulating the distal portion so that at least one therapeutic element is oriented toward a second renal vessel adjacent the first renal vessel; deploying the at least one therapeutic element to extend at least partially through the wall of the first renal vessel such that the at least therapeutic element extends toward the second renal vessel; and delivering a chemical agent through the plurality of therapeutic elements to modulate activity of at least one renal nerve adjacent to the first renal vessel and at least one renal nerve adjacent to the second renal vessel.

This application claims the benefit of U.S. Provisional Application No. 63/214,479, filed on Jun. 24, 2021, and entitled, “CHEMICAL NEUROMODULATION AGENT DELIVERY,” which is incorporated by reference herein in its entirety.

### TECHNICAL FIELD

The present technology is related to chemical neuromodulation. In particular, various examples of the present technology are related to chemical renal neuromodulation.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function can be considerably stimulated in disease states characterized by heightened sympathetic tone. For example, reduced renal blood flow and glomerular filtration rate as a result of renal sympathetic efferent stimulation may be a cornerstone of the loss of renal function in cardio-renal syndrome, (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, can have significant limitations including limited efficacy, compliance issues, side effects, and others.

### SUMMARY

The present technology is directed to devices, systems, and methods for neuromodulation, such as renal neuromodulation.

In some examples, the disclosure describes a method that includes positioning a distal portion of a neuromodulation catheter in a first renal vessel of a patient. The distal portion of the neuromodulation catheter may include a plurality of therapeutic elements arranged around a perimeter of the distal portion of the neuromodulation catheter. In a deployed configuration, the plurality of therapeutic elements may be configured to extend at least partially through a wall of the first renal vessel. The method also may include imaging the distal portion of the neuromodulation catheter to visualize positions of the plurality of therapeutic elements; manipulating the distal portion of the neuromodulation catheter so that at least one therapeutic element of the plurality of therapeutic elements is oriented toward a second renal vessel adjacent the first renal vessel; deploying the at least one therapeutic element to extend at least partially through the wall of the first renal vessel such that the at least therapeutic element extends toward the second renal vessel; and delivering a chemical agent through the plurality of therapeutic elements to modulate activity of at least one renal nerve adjacent to the first renal vessel and at least one renal nerve adjacent to the second renal vessel.

In some examples, the disclosure describes a method that includes positioning a distal portion of a neuromodulation catheter in a first renal vessel of a patient. The distal portion of the neuromodulation catheter may include a plurality of therapeutic elements arranged around a perimeter of the distal portion of the neuromodulation catheter. In a deployed configuration, the plurality of therapeutic elements may be configured to extend at least partially through a wall of the first renal vessel. The method also may include imaging the distal portion of the neuromodulation catheter to visualize positions of the plurality of therapeutic elements; manipulating the distal portion of the neuromodulation catheter so that a first therapeutic element of the plurality of therapeutic elements is oriented toward a second renal vessel adjacent the first renal vessel and a second therapeutic element of the plurality of therapeutic elements is oriented toward a third renal vessel adjacent the first renal vessel; deploying the first therapeutic element to extend at least partially through the wall of the first renal vessel such that the first therapeutic element extends toward the second renal vessel; deploying the second therapeutic element to extend at least partially through the wall of the first renal vessel such that the second therapeutic element extends toward the third renal vessel; and delivering a chemical agent through the plurality of therapeutic elements to modulate activity of at least one renal nerve adjacent to the first renal vessel, at least one renal nerve adjacent to the second renal vessel, and at least one renal nerve adjacent to the third renal vessel.

In some examples, the disclosure describes a method that includes positioning a distal portion of a neuromodulation catheter in a first renal vessel of a patient. The distal portion of the neuromodulation catheter may include a plurality of therapeutic elements arranged around a perimeter of the distal portion of the neuromodulation catheter. The method also may include imaging the distal portion of the neuromodulation catheter to visualize positions of the plurality of therapeutic elements; manipulating the distal portion of the neuromodulation catheter so that at least one therapeutic element of the plurality of therapeutic elements is oriented toward a second renal vessel adjacent the first renal vessel; and delivering neuromodulation energy via the plurality of therapeutic elements to modulate activity of at least one renal nerve adjacent to the first renal vessel and at least one renal nerve adjacent to the second renal vessel. A greater amount of neuromodulation energy may be delivered via the at least one therapeutic element of the plurality of therapeutic elements that is oriented toward a second renal vessel than is delivered via at least one other therapeutic element of the plurality of therapeutic elements.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout and wherein:

FIG. 1 is a partially schematic illustration of a neuromodulation system configured in accordance with some examples of the present disclosure.

FIG. 2 illustrates accessing of a renal artery and modulating renal nerves with the system of FIG. 1 in accordance with some examples of the present disclosure.

FIG. 3 is a conceptual illustration of an example sympathetic nervous system (SNS) and how the brain communicates with the body via the SNS.

FIG. 4 is an enlarged anatomic view of nerves innervating a left kidney to form the renal plexus surrounding the left renal artery.

FIG. 5 is an anatomic view of an example human body depicting neural efferent and afferent communication between the brain and kidneys.

FIG. 6 is a conceptual views of an example human body depicting neural efferent and afferent communication between the brain and kidneys.

FIG. 7 is an anatomic view of the arterial vasculature of a human.

FIG. 8 is an anatomic view of the venous vasculature of a human.

FIG. 9 is a conceptual diagram of an example neuromodulation catheter that includes a distal portion of an elongated shaft positioned within a main renal artery, in accordance with some examples of the present disclosure.

FIG. 10 is a conceptual diagram illustrating a schematic cross-sectional view of the neuromodulation catheter of FIG. 9 taken along line A-A, in accordance with some examples of the present disclosure.

FIG. 11 is a conceptual diagrams illustrating another schematic cross-sectional view of the neuromodulation catheter of FIG. 9 taken along line A-A with the example neuromodulation catheter in a different position and configuration than in FIG. 10, in accordance with some examples of the present disclosure.

FIG. 12 is a conceptual diagram illustrating an example neuromodulation catheter including a distal portion that includes a plurality of therapeutic elements, in accordance with some examples of the present disclosure.

FIG. 13 is a conceptual cross-sectional diagram of a handle of a neuromodulation catheter, in accordance with some examples of the present disclosure.

FIG. 14 is a conceptual diagram of another example neuromodulation catheter that includes a handle that enables allows a clinician to control the deployment distance or extension length of different needles independently, in accordance with some examples of the present disclosure.

FIG. 15 is a conceptual diagram illustrating an example neuromodulation catheter that includes a proximal portion that enables control of an amount of chemical agent delivered to at least one needle separately from one or more other needles, in accordance with some examples of the present disclosure.

FIG. 16 is a conceptual diagram illustrating an example neuromodulation catheter that includes a plurality of therapeutic elements oriented in a generally distal direction relative to neuromodulation catheter, in accordance with some examples of the present disclosure.

FIG. 17 is a conceptual diagram illustrating an example neuromodulation catheter that includes a plurality of therapeutic elements and an expandable centering element, in accordance with some examples of the present disclosure.

FIG. 18 is a conceptual diagram illustrating a schematic cross-sectional view of an example neuromodulation catheter that includes a plurality of therapeutic elements configured to deliver neuromodulation energy, in accordance with some examples of the present disclosure.

FIG. 19 is a conceptual diagram illustrating a schematic cross-sectional view of an example neuromodulation catheter that includes a plurality of therapeutic elements configured to deliver neuromodulation therapy to three renal vessels from a single deployment location, in accordance with some examples of the present disclosure.

FIG. 20 is a flow diagram illustrating an example technique of performing neuromodulation of renal nerves, in accordance with some examples of this disclosure.

FIG. 21 is a flow diagram illustrating an example technique of performing neuromodulation of renal nerves, in accordance with some examples of this disclosure.

FIG. 22 is a flow diagram illustrating an example technique of performing neuromodulation of renal nerves, in accordance with some examples of this disclosure.

### DETAILED DESCRIPTION

The present technology is directed to devices, systems, and methods for neuromodulation, such as renal neuromodulation, using chemical agents.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

Renal neuromodulation, such as renal denervation, may be accomplished using one or more of a variety of treatment modalities, including radio frequency (RF) energy, microwave energy, ultrasound energy, a chemical agent, or the like. When using a chemical agent, a neuromodulation catheter may be delivered to a renal vessel, such as a renal artery, of a patient. The neuromodulation catheter may include at least one port or needle through which the chemical agent is delivered. The chemical agent may be selected to modulate activity of one or more renal nerves adjacent to the renal artery in which the neuromodulation catheter is positioned. For example, the chemical agent may be a neurotoxic chemical selected to chemically ablate the one or more renal nerves near the renal artery.

In some examples, a patient may benefit from renal neuromodulation at multiple locations. For example, some patients may have more than one renal artery that extends from the aorta toward a single kidney. One of these multiple arteries may be referred to as a main renal artery, while the one or more other arteries may be referred to as an accessory renal artery. Some patients have one accessory renal artery, while some patients have two or more accessory renal arteries. Accessory renal arteries are not redundant to the main renal artery; rather, blood flows through the accessory renal artery or arteries to different portions of the kidney, and different nerves innervate the wall and perivascular space of the accessory artery or arteries and the main renal artery. Thus, the clinician may desire to treat both the main renal artery and the at least one accessory renal artery. If treated separately, this may increase a total treatment time, which may increase cost of the procedure.

In accordance with techniques of this disclosure, a neuromodulation catheter may include one or more features configured to enable treatment of tissues adjacent to second vessel (such as an accessory renal artery) while the neuromodulation catheter is positioned within a first vessel (such as a main renal artery). Alternatively, or additionally, the disclosure describes techniques for using a neuromodulation catheter to treat tissues adjacent to a first vessel in which the neuromodulation catheter is positioned (such as a main renal artery) and adjacent to a second vessel (such as an accessory renal artery). In some examples, rather than the neuromodulation catheter being positioned in a main renal artery adjacent an accessory renal artery being substantially simultaneously treated, the neuromodulation catheter may be positioned in an accessory renal artery and another accessory renal artery and/or a main renal artery may be substantially simultaneously treated. For instance, the main renal artery or an accessory renal artery of a patient may be undersized or anatomically hard to access, and the neuromodulation catheter may be positioned in an easier to access or larger vessel and used to substantially simultaneously treat the vessel in which the neuromodulation catheter is positioned and the adjacent undersized or anatomically hard to access vessel.

Techniques for using a neuromodulation catheter to substantially simultaneously treat tissues adjacent to a first renal vessel (such as a main renal artery) and a second renal vessel (such as an accessory renal artery) may facilitate orientation of at least one therapeutic element toward the second renal vessel. For instance, a distal portion of the neuromodulation catheter may be positioned in the first renal vessel. An imaging device, such as a fluoroscopic C-arm machine, may be used to image the distal portion of the neuromodulation catheter and surrounding physiology of the body of the patient. The imaging device may be used to visualize positions of the plurality of therapeutic elements, positions of the first renal vessel and a second renal vessel, or both. For instance, the C-arm of the fluoroscopic C-arm machine may be manipulated so that a longitudinal axis of the first renal vessel and a longitudinal axis of the second renal vessel are substantially in the plane of the image.

The clinician may manipulate the distal portion of the neuromodulation catheter, for example, by rotating a proximal portion of the neuromodulation catheter about a longitudinal axis of the neuromodulation catheter, so that at least one therapeutic element is oriented toward the second renal vessel. For instance, a neuromodulation catheter may include a distal portion configured to be positioned in a renal vessel of a patient, such as a main renal artery. The distal portion may include a plurality of therapeutic elements arranged around an outer perimeter (e.g., referred to herein as a circumference, though the catheter may have other geometries in other examples) of the distal portion of the catheter. For example, the neuromodulation catheter may include three or more therapeutic elements arranged around a circumference of the distal portion of the catheter. In some implementations, the therapeutic elements may each include a needle. The needle(s) may be configured to be radially extended from the neuromodulation catheter to pierce a wall of the renal vessel. In some examples, an extension distance of each needle may be controlled independently, such that a needle oriented generally toward a second renal vessel (such as an accessory renal artery) may be extended further than at least one other needle that is not oriented generally toward the second renal vessel. This may enable more selective delivery of the chemical agent nearer to the accessory article than if all the needles are only collectively controlled and extended substantially the same distance. Consequently, this may increase influence of the chemical agent on renal nerves surrounding or near the second renal vessel.

Additionally, or alternatively, the neuromodulation catheter may enable independent control of an amount of chemical agent injected through each needle. This may allow additional chemical agent to be delivered through the needle oriented generally toward the second renal vessel while delivering different amounts or no chemical agent through other needles. Consequently, this may increase influence of the chemical agent on renal nerves surrounding or near the second renal vessel.

In other implementations, the therapeutic elements may each include an electrode for delivery of RF or microwave energy, an ultrasound transducer for delivery of ultrasound energy, or another energy delivery device. By orienting at least one therapeutic element of the plurality of therapeutic elements toward a second renal vessel adjacent the first renal vessel, the techniques described herein may enable directed delivery of energy toward the second renal vessel adjacent the first renal vessel while the neuromodulation catheter is positioned in the first renal vessel. In some examples, the amount of energy delivered by each therapeutic element may be controlled (e.g., independently) so that a greater amount of energy may be delivered via the therapeutic element oriented toward the second renal vessel.

In some examples, the techniques described herein may be extended to more than two vessels. For instance, some patients include three renal arteries that extend from the aorta toward a single kidney. The techniques described herein may be used to position a neuromodulation catheter in one of the three vessels and orient therapeutic elements toward the other two vessels. In this way, three vessels may be treated with the catheter in a single location within one of the vessels.

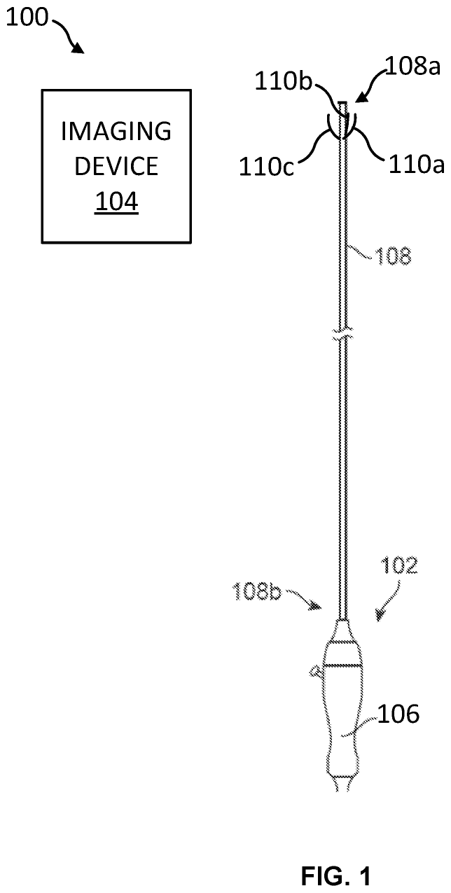

FIG. 1 is a partially schematic illustration of a neuromodulation system 100 (“system 100”) configured in accordance with some examples of the present technology. As shown in FIG. 1, system 100 includes a neuromodulation catheter 102 and an imaging device 104. Neuromodulation catheter 102 includes a handle 106 and an elongated shaft 108 attached to the handle 106. The elongated shaft 108 may include a distal portion 108a and a proximal portion 108b. Distal portion 108a of neuromodulation catheter 102 includes a plurality of therapeutic elements 110a-110c (“therapeutic elements 110”). Therapeutic elements 110 may be positioned around (e.g., distributed around) a circumference of distal portion 108a.

Although distal portion 108a is shown in FIG. 1 as including three therapeutic elements 110 positioned around a circumference of distal portion 108a at a single longitudinal position along elongated shaft 108, in other examples, distal portion 108a may include any number of therapeutic elements 110. For example, distal portion 108a may include two, three, four, or more therapeutic elements 110 positioned around a circumference of distal portion 108a at a single longitudinal position. As another example, distal portion 108a may include positioned two, three, four, or more therapeutic elements 110 positioned around a circumference of distal portion 108a at each of multiple longitudinal positions along distal portion 108a. In examples in which distal portion 108a includes therapeutic elements 110 positioned at different longitudinal positions, each longitudinal position may include one or more therapeutic element 110, and each longitudinal position may include the same number of therapeutic elements 110, or one longitudinal position may include a different number of therapeutic elements 110 than one or more other longitudinal positions.

Elongated shaft 108 may have any suitable outer diameter, and the diameter can be constant along the length of elongated shaft 108 or may vary along the length of elongated shaft 108. In some examples, elongated shaft 108 can be 2, 3, 4, 5, 6, or 7 French or another suitable size.

Distal portion 108a of elongated shaft 108 is configured to be moved within a lumen of a human patient to locate therapeutic elements 110 at a target site within or otherwise proximate to the lumen. For example, elongated shaft 108 may be configured to position therapeutic elements 110 within a blood vessel, a ureter, a duct, an airway, or another naturally occurring lumen within the human body. In certain examples, intravascular delivery of the therapeutic elements 110 includes percutaneously inserting a guidewire (not shown) into a body lumen of a patient and moving elongated shaft 108 and/or therapeutic elements 110 along the guidewire until therapeutic elements 110 reaches a target site (e.g., a renal artery). For example, the distal end of elongated shaft 108 may define a passageway for engaging the guidewire for delivery of therapeutic elements 110 using over-the-wire (OTW) or rapid exchange (RX) techniques. In other examples, neuromodulation catheter 102 can be a steerable or non-steerable device configured for use without a guidewire. In still other examples, neuromodulation catheter 102 can be configured for delivery via a guide catheter or sheath (not shown), or other guide device.

Once at the target site, therapeutic elements 110 can be configured to deliver therapy, such as RF energy, microwave energy, ultrasound energy, a chemical agent, or the like to provide or facilitate neuromodulation therapy at the target site. For ease of description, the following discussion will be primarily focused on delivering a chemical agent, such as a neurotoxic chemical, in which examples therapeutic elements 110 include needles. It will be understood, however, that therapeutic elements 110 may include elements or structures configured to deliver other types of therapy. For example, therapeutic elements 110 may include needle electrodes configured to deliver RF energy for RF ablation of nerves near the lumen in which neuromodulation catheter 102 is positioned.

In examples in which neuromodulation catheter 102 is configured to deliver a chemical agent, therapeutic elements 110 may include needles configured to be deployed to extend radially from distal portion 108a and at least partially pierce a wall of the lumen in which distal portion 108a is positioned. The needles may extend to and/or through the intima, media, and/or adventitia of the wall and be configured to deliver the chemical agent to the adventitia and/or peri-adventitia, in which renal nerves are located. By having therapeutic elements 110 located around a circumference of distal portion 108a, neuromodulation catheter 102 may be used to deliver the chemical agent around a circumference of the lumen in which distal portion 108a is positioned. Again, while a circumference of the lumen is generally referred to herein, the lumen may not be perfectly circular in cross-section and may have any suitable geometry in cross-section.

System 100 also includes an imaging device 104. Imaging device 104 may include any suitable imaging modality configured to image distal portion 108a of elongated shaft 108 while elongated shaft 108 is within the lumen (such as a renal artery) and/or being advanced to the lumen. In some examples, imaging device 104 includes a computed tomography (CT) machine, a fluoroscopy machine, an intravascular ultrasound (IVUS) machine, an optical coherence tomography (OCT) machine, an intracardiac echocardiography (ICE) machine, or another suitable guidance modality, or combinations thereof. In examples in which imaging device 104 includes a fluoroscopic imaging device, the fluoroscopic imaging device may include a fluoroscopic c-arm machine.

In examples in which imaging device 104 includes a fluoroscopic c-arm, the fluoroscopic c-arm may be manipulated to orient an image plane relative to physiology of the patient. For instance, the fluoroscopic c-arm may be manipulated to orient the image plane so that at least a portion of a length of a first renal vessel (such as a main renal artery in which distal portion 108a is positioned) and at least a portion of a length of a second, adjacent renal vessel (such as an accessory renal artery extending generally parallel to the main renal artery) are both substantially in the plane of the image captured by imaging device 104. This may facilitate manipulation of distal portion 108a to orient at least one of therapeutic elements 110 in a desired orientation relative to the second renal vessel. Further, imaging device 104 may be used to image relative positions and orientations of therapeutic elements 110 at the target site.

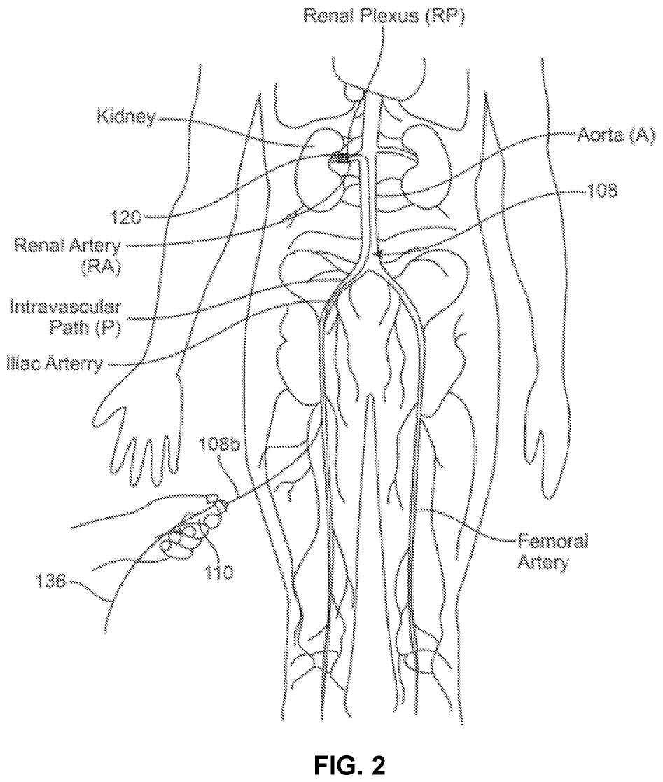

FIG. 2 (with additional reference to FIG. 1) illustrates gaining access to renal nerves of an example patient in accordance with some examples of the present technology. Neuromodulation catheter 102 provides access to the renal plexus RP through an intravascular path P, such as a percutaneous access site in the femoral (illustrated), brachial, radial, or axillary artery to a targeted treatment site within a respective renal artery RA. By manipulating proximal portion 108b of elongated shaft 108 from outside the intravascular path P, a clinician may advance at least distal portion 108a of elongated shaft 108 through the sometimes tortuous intravascular path P and remotely manipulate distal portion 108a (FIG. 1) of elongated shaft 108.

In the example illustrated in FIG. 2, therapeutic elements 110 are delivered intravascularly to the treatment site using a guidewire 136 in an OTW technique. As noted previously, the neuromodulation assembly 120 may define a passageway for receiving guidewire 136 for delivery of the neuromodulation catheter 102 using either an OTW or a RX technique. At the treatment site, guidewire 136 can be at least partially withdrawn or removed, and therapeutic elements 110 can transform or otherwise be moved to a deployed arrangement for delivering a chemical agent. In other examples, therapeutic elements 110 may be delivered to the treatment site within a different guide device, such as a guide sheath (not shown), with or without using guidewire 136. In examples in which the system includes a guide sheath, when therapeutic elements 110 are at the target site, the guide sheath may be at least partially withdrawn or retracted and therapeutic elements 110 may be transformed into the deployed arrangement. For example, at least a portion of therapeutic elements 110 may be self-expandable such that they expand to the deployed arrangement upon being released from the guide sheath. In still other examples, elongated shaft 108 may be steerable itself such that therapeutic elements 110 may be delivered to the treatment site without the aid of guidewire 136 and/or a guide sheath.

Imaging device 104 (FIG. 1) may enable image guidance, e.g., computed tomography (CT), fluoroscopy, intravascular ultrasound (IVUS), optical coherence tomography (OCT), intracardiac echocardiography (ICE), or another suitable guidance modality, or combinations thereof, to be used to aid the clinician's positioning and manipulation of distal portion 108a and therapeutic elements 110. For example, a fluoroscopy system (e.g., including a flat-panel detector, x-ray, or c-arm) can be rotated to accurately visualize and identify the target treatment site. In other examples, the target treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering therapeutic elements 110. Further, in some examples, image guidance components (e.g., IVUS, OCT) may be integrated with neuromodulation catheter 102 and/or run in parallel with neuromodulation catheter 102 to provide image guidance during positioning of therapeutic elements 110. For example, image guidance components (e.g., IVUS or OCT) can be coupled to therapeutic elements 110 to provide three-dimensional images of the vasculature proximate the target site to facilitate positioning or deploying therapeutic elements 110 within the target renal blood vessel.

Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced, thermally-induced, chemically-induced, or induced in another suitable manner or combination of manners at one or more suitable target sites during a treatment procedure. The target site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery.

As described above, some patients may have multiple arteries extending from the aorta (A) to a single kidney (i.e., a right kidney or a left kidney). One of these multiple arteries may be referred to as a main renal artery, while the other(s) of these multiple arteries may be referred to as accessory renal arteries. In patients that have multiple arteries extending from the aorta (A) to a single kidney, some renal nerves may travel along or approach a main renal artery and some nerves may travel along or approach the accessory renal artery or arteries. As such, a treatment procedure may include treating nerves along or approaching the main renal artery and the accessory renal artery or arteries.

Treatment procedures that treat at multiple target sites may require repositioning of neuromodulation catheter 102, which may increase the procedure time, complexity, and cost. Thus, combining treatments at multiple target sites into a single treatment step may save time, simplify the procedure, and reduce cost to the patient. As described above, neuromodulation catheter 102 is configured to enable treatment of nerves along or approaching a first renal vessel (such as a main renal artery) and nerves along or approaching a second renal vessel (such as an accessory renal artery) in a single treatment step.

The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of treatment devices and associated methods for achieving renal neuromodulation via intravascular access and impose specific design requirements for such devices. Specific design requirements may include accessing the renal artery, positioning therapeutic elements 110 within the renal artery and relative to other physiological structures (such as an accessory renal artery), delivering the chemical agent to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

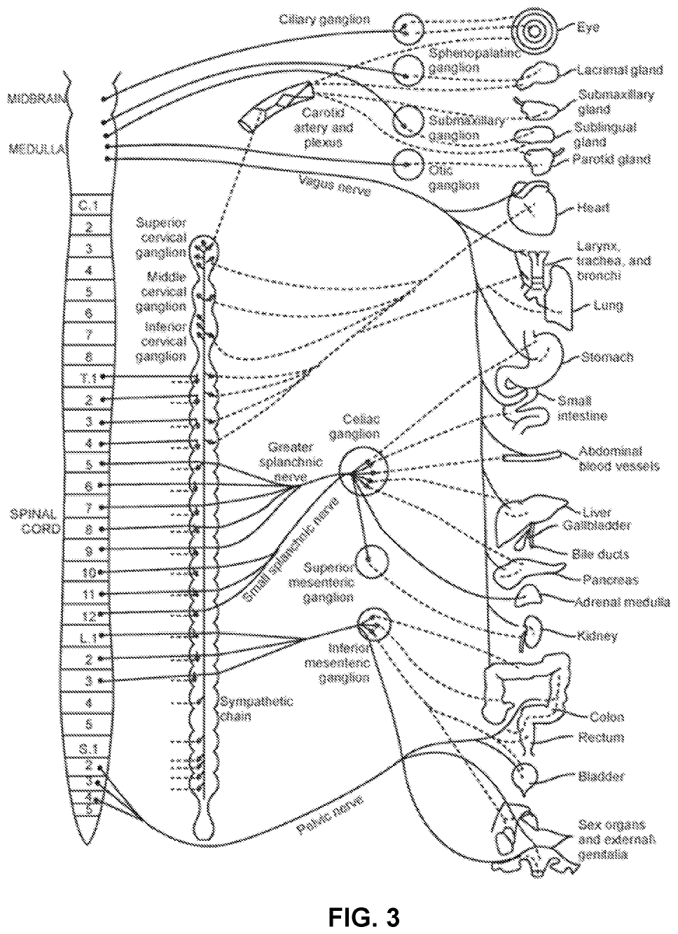

As shown in FIG. 3, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

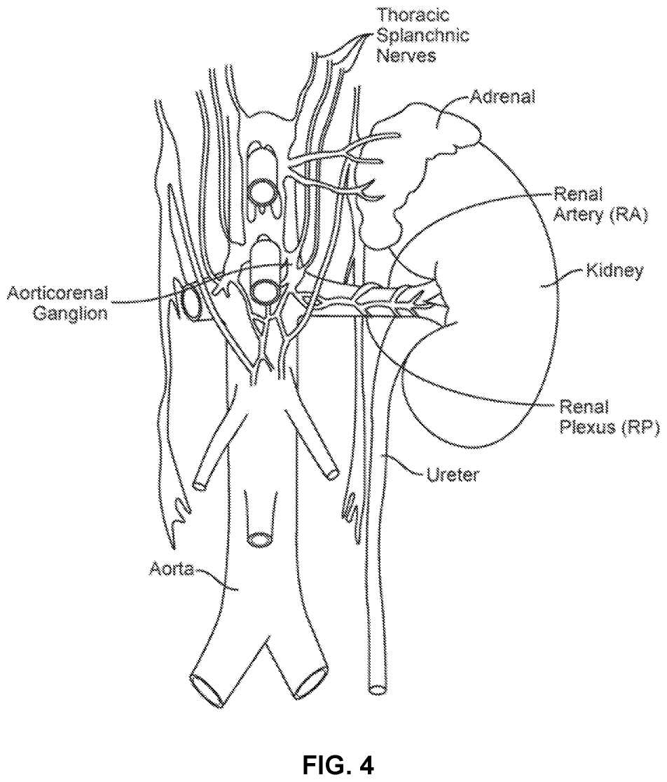

As FIG. 4 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery until it arrives at the substance of the kidney. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); or raise blood pressure. Afferent messages carry signals from various organs and sensory receptors in the body to other organs and, particularly, the brain.

Hypertension, heart failure, and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease in some patients are characterized by heightened sympathetic nervous activation. In patients with end stage renal disease, plasma levels of norepinephrine above the median have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus and the renal tubules. Stimulation of the renal sympathetic nerves causes increased renin release, increased sodium (Nat) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects and others.

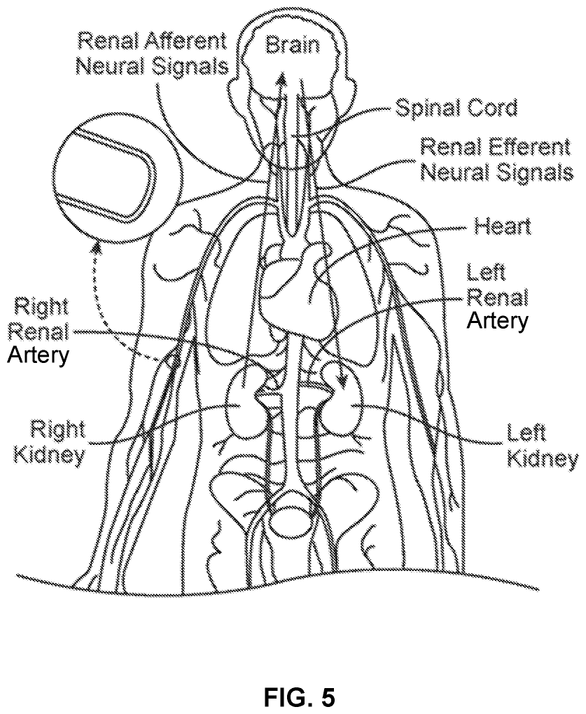

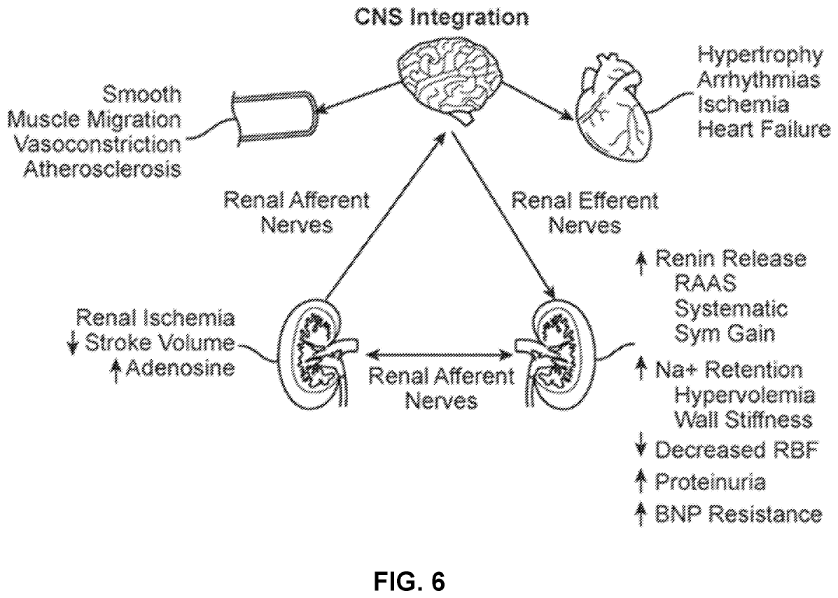

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 5 and 6, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 5. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

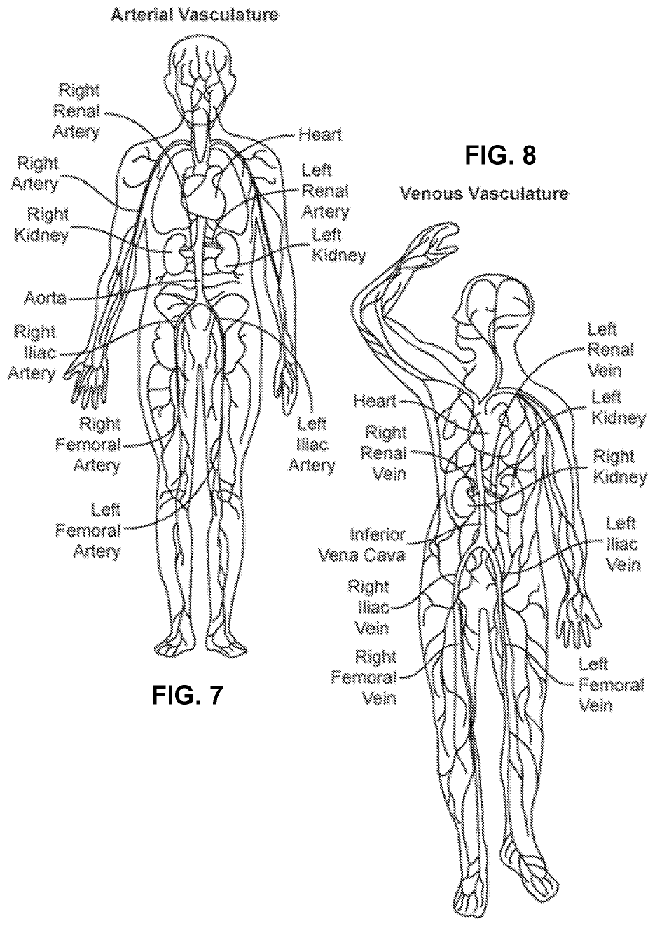

In accordance with the present technology, neuromodulation of a left and/or right renal plexus (RP), which is intimately associated with a left and/or right renal artery, may be achieved through intravascular access. As FIG. 7 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 8 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

Even after accessing a renal artery and facilitating stable contact between neuromodulatory apparatus and a luminal surface of the artery, nerves in and around the adventia of the artery should be safely modulated via the neuromodulatory apparatus. Effectively applying chemical treatment from within a renal artery is non-trivial given the potential clinical complications associated with such treatment. For example, the intima and media of the renal artery may be vulnerable to chemical injury. As discussed in greater detail below, the intima-media thickness separating the vessel lumen from its adventitia means that target renal nerves may be multiple millimeters distant from the luminal surface of the artery. The chemical agent should be delivered to the target renal nerves to modulate the target renal nerves without excessively adversely impacting the vessel wall to the extent that the vessel wall is potentially affected to an undesirable extent.

The neuromodulatory apparatus should also be configured to allow for adjustable positioning and repositioning of the therapeutic elements 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that therapeutic elements 110 may intimately contact the vessel wall and/or extend at least partially through the vessel wall. Renal artery vessel diameter, D_{RA}, typically is in a range of about 2-10 millimeters (mm), with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the renal artery) to avoid non-target tissue and anatomical structures such as anatomical structures of the digestive system or psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

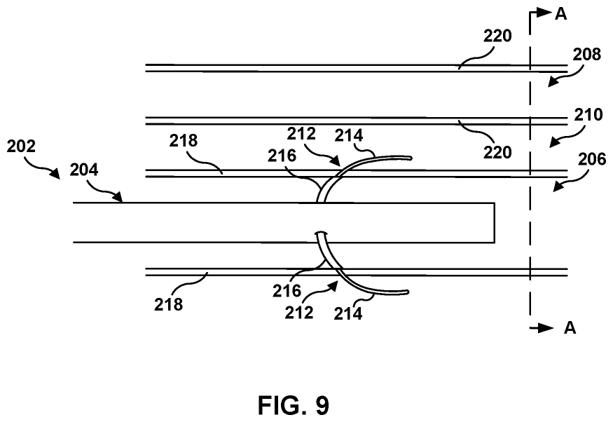

FIG. 9 is a conceptual diagram of an example neuromodulation catheter 202 that includes a distal portion 204 of an elongated shaft of catheter 202 positioned within a first renal artery 206. Neuromodulation catheter 202 is an example of neuromodulation catheter 102 of FIG. 1. In some instances, distal portion 204 may be positioned in a distal aspect of a main renal artery. FIG. 9 also shows a second renal artery 208 adjacent to first renal artery 206 and separated by tissue 210. Second renal artery 208 may include an accessory renal artery. In other examples, first renal artery 206 may be an accessory renal artery and second renal artery 208 may include another accessory renal artery or a main renal artery. First renal artery 206 includes wall 218 and second renal artery 208 includes wall 220. As shown in FIG. 9, neuromodulation catheter 202 includes a plurality of therapeutic elements 212 positioned circumferentially about distal portion 204.

Neuromodulation catheter 202 is configured to deliver a chemical agent, such as a neurotoxic chemical, through the plurality of therapeutic elements 212. The chemical agent may be selected to neuromodulate (e.g., chemically ablate) nerve tissue of the renal plexus adjacent to first renal artery 206 and second renal artery 208. The chemical agent may include, for example, an alcohol, such as ethanol; distilled water; hypertonic saline; hypotonic saline; phenol; glycerol; lidocaine; bupivacaine; tetracaine; benzocaine; guanethidine; botulinum toxin; another appropriate neurotoxic fluid; or combinations thereof. In some examples, the chemical agent may be heated or cooled to additionally or alternatively thermally ablate nerve tissue of the renal plexus adjacent to first renal artery 206 and second renal artery 208.

In the example shown in FIG. 9, two therapeutic elements 212 are shown. However, neuromodulation catheter 202 includes three therapeutic elements 212, one of which is not shown because it is occluded by the body of neuromodulation catheter 202. In other examples, neuromodulation catheter 202 includes only one or two therapeutic elements 212 or more than three therapeutic elements 212. Each therapeutic element 212 includes a corresponding needle 214 and, in some, but not all, examples, a corresponding guide tube 216. Each guide tube 216 is configured to, for example, define a pathway through which a corresponding needle 214 may traverse to reach a target site for delivery of a therapy.

Therapeutic elements 212 are configured to be carried by neuromodulation catheter 202 in a delivery configuration as distal portion 204 of neuromodulation catheter 202 is advanced through vasculature of the patient to first renal artery 206. Therapeutic elements 212 are also configured to transform to a deployed configuration, which is shown in FIG. 9. For example, in the delivery configuration, needles 214 may be withdrawn into guide tubes 216 and guide tubes 216 may be retracted within neuromodulation catheter 202. To transition to the deployed configuration from the delivery configuration, guide tubes 216 may be advanced out of neuromodulation catheter 202 to extend radially outwards relative to neuromodulation catheter 202 and contact the wall 218 of first renal artery 206, as shown in FIG. 9. After guide tubes 216 have been advanced out of neuromodulation catheter 202 to contact the wall 218, needles 214 may be advanced out of guide tubes 216 at least partially through wall 218 (e.g., only partially through wall 218 or completely through wall 218). Alternatively, needles 214 may be fixed relative to guide tubes 216, such that guide tubes 216 and needles 214 are extended out of neuromodulation catheter 202 together and the exposed length of needles 214 out of guide tubes 216 is fixed. To transition back to the delivery configuration, guide tubes 216 and needles 214 may be retracted within neuromodulation catheter 202.

As another example, in the delivery configuration, needles 214 may be withdrawn into guide tubes 216 and guide tubes 216 may be urged against an outer surface of neuromodulation catheter 202 using a guide sheath (not shown in FIG. 9). To transition to the deployed configuration, the guide sheath may be withdrawn proximally relative to neuromodulation catheter 202, which allows guide tubes 216 to expand radially outward. As such, in some examples, guide tubes 216 may be formed of a resilient material, such as a shape memory material, which is self-expanding upon being released from the guide sheath. Once guide tubes 216 have expanded to contact wall 218, needles 214 may be advanced out of guide tubes 216 at least partially through wall 218.

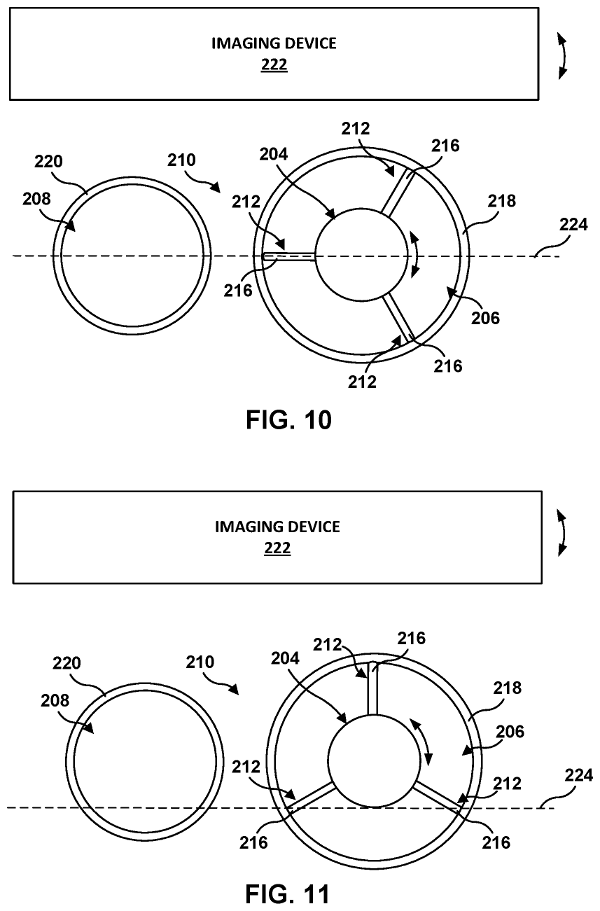

As shown in FIG. 9, at least one of therapeutic elements 212 may be oriented toward second renal artery 208 when the at least one therapeutic element 212 is in the deployed configuration. FIGS. 10 and 11 are conceptual diagrams illustrating cross-sectional views of FIG. 9 taken along line A-A (e.g., in a directly orthogonal to a longitudinal axis of a shaft of catheter 202) with neuromodulation catheter 202 in different positions and configurations. Like FIG. 9, FIGS. 10 and 11 depict distal portion 204 of neuromodulation catheter 202 within first renal artery 206 and an adjacent second renal artery 208. First renal artery 206 and second renal artery 208 are separated by tissue 210. Neuromodulation catheter 202 includes a plurality of therapeutic elements 212, in this example three neuromodulation elements 212, arranged about a circumference of distal portion 204. In the examples shown in FIGS. 10 and 11, guide tubes 216 are in a deployed position, but needles 214 have not been deployed and thus are not shown. FIGS. 10 and 11 also conceptually illustrate an imaging device 222 that captures an image at an image plane 224. Imaging device 222 is an example of imaging device 104 of FIG. 1.

As shown in FIGS. 10 and 11, therapeutic elements 212 may be arranged around a circumference of distal portion 204. For example, therapeutic elements 212 may be spaced substantially equally around the circumference of distal portion 204. In an example with three therapeutic elements 212, the therapeutic elements 212 may be arranged at intervals of about 120°.

Imaging device 222 is configured to image distal portion 204 and surrounding physiological environment of a patient, including at least portions of first renal artery 206 and second renal artery 208. Imaging device 222 may be manipulable to orient image plane 224 relative to distal portion 204, first renal artery 206, and second renal artery 208. For instance, in examples in which imaging device 222 is a fluoroscopic c-arm machine, a clinician, manually or with the aid of a device, can manipulate the c-arm to control image plane 224 relative to distal portion 204, first renal artery 206, and second renal artery 208.

Imaging device 222 may be manipulated so that at least a portion of first renal artery 206 and second renal artery 208 lie substantially within image plane 224. For example, a portion of first renal artery 206 may define a first longitudinal axis and a portion of second renal artery 208 may define a second longitudinal axis. A clinician may manipulate imaging device 222 so that imaging plane 224 is substantially parallel to the first longitudinal axis and the second longitudinal axis. Further, in some implementations, a clinician may control a focal depth of imaging device 222 so that image plane 224 is within the portion of first renal artery 206 and the portion of second renal artery 208. This may facilitate placement and orientation of distal portion 204 within first renal artery 206 with at least one of therapeutic elements 212 oriented toward second renal artery 208.

As shown in FIGS. 10 and 11, a clinician may manipulate distal portion 204 so that at least one of therapeutic elements 212 is oriented toward second renal artery 208. In FIG. 10, one therapeutic element 212 is oriented directly toward second renal artery 208. In FIG. 11, distal portion 204 has been rotated about the longitudinal axis of the sheath of catheter 202 so that one therapeutic element 212 is oriented toward (but not directly toward) second renal artery 208. In the example shown in FIG. 11, portions of two therapeutic elements 212 are in the image plane 224, which may help the clinician visualize the orientation of therapeutic elements 212 (including the relative in- or out-of-the-image-plane orientation) relative to second renal artery 208.

To facilitate medical imaging of therapeutic elements 212, guide tubes 216 may include or be formed from a radiopaque material. For example, guide tubes 216 may be formed from a radiopaque metal, such as tantalum, gold, platinum, or the like. As another example, guide tubes 216 may be formed from a polymer and include a radiopaque marker element, such as a ring or band, formed from a radiopaque metal, such as tantalum, gold, platinum, barium, tungsten, or the like. As a further example, guide tubes 216 may be formed from a polymer filled with a radiopaque powder, such as tantalum, gold, platinum, barium, tungsten, or the like. In examples in which guide tubes 216 include a plastic, the plastic may include a polyamide, a polyurethane, or the like; or a multilayer construction that includes an inner layer of polyamide and an outer layer of polyurethane.

By imaging distal portion 204, the clinician may determine the positions of therapeutic elements 212 relative to each other and relative to second renal artery 208. This enables a clinician to manipulate distal portion 204 (e.g., by manipulating a proximal portion of neuromodulation catheter 202 or a handle of neuromodulation catheter 202) so that at least one of therapeutic elements 212 is oriented toward second renal artery 208. Once neuromodulation catheter 202 is oriented as desired, the clinician can deploy needles 214, one or more of which will be oriented toward second renal artery 208.

Deploying needles 214 with at least one needle oriented toward second renal artery 208 may result in a chemical agent being directed out of the at least one needle oriented toward second renal artery 208 to tissue 210 between first renal artery 206 and second renal artery 208 and, in some examples, to tissue surrounding second renal artery 208. This may be more effective when at least one needle is oriented toward second renal artery 208 than if none of needles 214 are oriented toward second renal artery 208. For example, a greater amount of chemical agent may reach tissue 210 and/or tissue surrounding second renal artery 208 when at least one needle is oriented toward second renal artery 208. This may enable treatment of nerves along or approaching first renal artery 206 and second renal artery 208 in a single step.

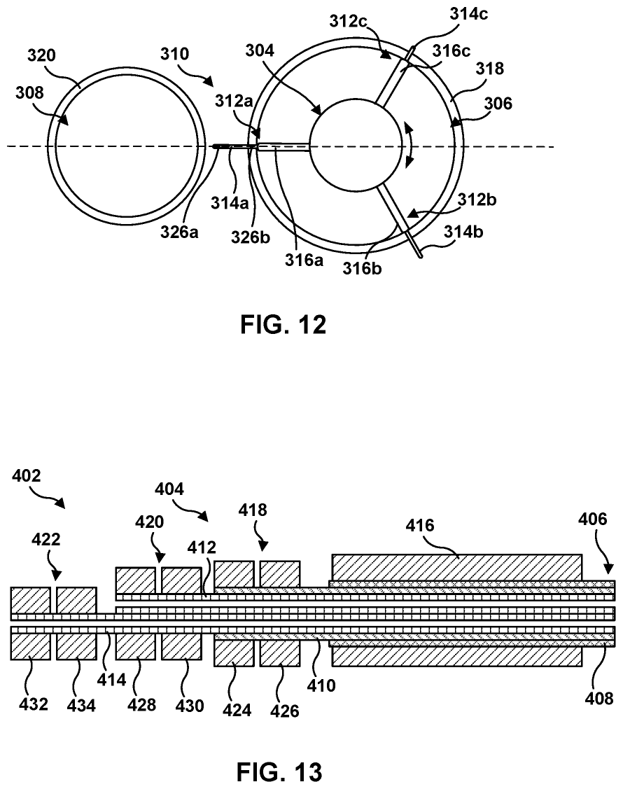

In some examples, a neuromodulation catheter may include one or more additional features that facilitate treatment of a first renal vessel and a second renal vessel in a single treatment step. For example, FIG. 12 is a conceptual diagram illustrating an example neuromodulation catheter 302 including a distal portion 304 that includes a plurality of therapeutic elements 312a-312c (“therapeutic elements 312”). Neuromodulation catheter 302 is an example of neuromodulation catheter 102 of FIG. 1. Distal portion 304 is positioned in a main renal artery 306, which is adjacent to an auxiliary renal artery 308. First renal artery 306 includes wall 318, and second renal artery 308 includes wall 320. First renal artery 306 and second renal artery 308 are separated by tissue 310. As described above, first renal artery 306 may include a main renal artery and second renal artery 308 may include an accessory renal artery, or first renal artery 306 may include an accessory renal artery and second renal artery 308 may include another accessory renal artery or a main renal artery.

Like therapeutic elements 212 of neuromodulation catheter 202, each of therapeutic elements 312 includes a corresponding needle of needles 314a-314c (“needles 314”) and a corresponding guide tube of guide tubes 316a-316c (“guide tubes 316”). Unlike therapeutic elements 212 of neuromodulation catheter 202, at least one needle of needles 314 is configured to be extended a different deployment distance than one or more other needle of needles 314.

In some examples, each needle of needles 314 is fixed relative to the corresponding guide tube of guide tubes 316. For instance, needle 312a is fixed relative to guide tube 316a, needle 312b is fixed relative to guide tube 316b, and needle 314c is fixed relative to guide tube 316c. At least one needle of needles 314 may be fixed with a different exposed length (and thus a different deployment distance) than one or more other needle of needles 314.

In the example illustrated in FIG. 12, needle 314a has the greatest exposed length and deployment distance (e.g., measured in a radial direction relative to an outer surface of distal portion 304), while needle 314c has the shortest exposed length and deployment distance. Needle 314b has an intermediate exposed length and deployment distance. In other examples, needles 314b and 314c may have the same exposed length and deployment distance. Because needle 314a has the greatest exposed length and deployment distance, a clinician may manipulate distal portion 304 so that therapeutic element 312a is oriented toward second renal artery 308. This results in needle 314a delivering the chemical agent nearer to second renal artery 308 than if needle 314b or needle 314c were oriented toward second renal artery 308. This may result in the chemical agent being introduced into tissue closer to and possibly surrounding second renal artery 308, facilitate neuromodulation of renal nerves adjacent to second renal artery 308.

In other examples, each needle of needles 314 is independently extendable and retractable. For example, each needle of needles 314 may be coupled to an independent control element at a proximal end of neuromodulation catheter 302 or attached to neuromodulation catheter 302. This allows a clinician to control the deployment distance or extension length of different needles of needles 314 independently.

In some implementations, each needle of needles 314 may include multiple ports for delivery of the chemical agent to tissue. For example, as shown in FIG. 12, needle 314a includes a first port 326a at a distal portion of needle 314a and a second port 326b at a more proximal portion of needle 314a. For instance, second port 326b may be located approximately 4 mm from the distal tip of needle 314a. First port 326a may be exposed when needle 314a is less fully extended from guide tube 316a, while second port 326b may only be exposed when needle 314a is more fully extended from guide tube 316a. This may enable delivery of chemical agent to both near second renal artery 308 and near first renal artery 306 by needle 314a and facilitate substantially simultaneous treatment of renal nerves adjacent to first renal artery 306 and second renal artery 308. Although ports are not shown on other needles 314b and 314c, any needles described herein may include a similar or different arrangement of one or more needle ports.

For example, FIG. 13 is a conceptual cross-sectional diagram of a handle 404 of a neuromodulation catheter 402, which is any example of catheters 102 and 304 of FIGS. 1 and 12, respectively. Handle 404 is coupled to or integral with elongated shaft 406, which includes an outer body portion 408, a first inner body portion 410, a second inner body portion 412, and a third inner body portion 414. Handle 404 includes a first handle portion 416 coupled to outer body portion 408, a second handle portion 418 coupled to first inner body portion 410, a third handle portion 420 coupled to second inner body portion 412, and a third handle portion 422 coupled to third inner body portion 414.

Outer body portion 408 and first handle portion 416 may be configured to control overall axial and rotational movement of elongated shaft 406. As such, a clinician may manipulate first handle portion 416 to apply an axial pushing force to elongated shaft 406 to advance a distal portion of elongated shaft 406 to a target therapy site and to rotate the distal portion of elongated shaft 406 about its longitudinal axis to orient therapeutic elements carried by the distal portion of elongated shaft 406.

Second handle portion 418 is coupled to first inner body portion 410, which may be coupled to guide tubes of the therapeutic elements and configured to transfer axial forces applied to second handle portion 418 to the guide tubes to extend and retract the guide tubes from the distal portion of elongated shaft 406. The distance between a distal end of second handle portion 418 and a proximal end of first handle portion 416 may define an extension length for the guide tubes.

In some examples, second handle portion 418 includes a first section 424 and a second section 426. First and second sections 424 and 426 may be selectively movable relative to each other to define an extension length for the guide tubes. For example, axial movement of second section 426 away from first section 424 (followed by locking or otherwise restraining first and second sections 424 and 426 relative to each other) may reduce a distance that the guide tubes can be extended, while axial movement of second section 426 toward first section 424 (followed by locking or otherwise restraining first and second sections 424 and 426 relative to each other) may increase a distance that the guide tubes can be extended. In some examples, first and second sections 424 and 426 are threaded to each other so that relative rotation causes relative axial movement between first and second sections 424 and 426.

Third handle portion 420 is coupled to second inner body portion 412, which may be coupled to at least one needle of the therapeutic elements and configured to transfer axial forces applied to third handle portion 420 to the at least one needle to extend and retract the at least one needle from the guide tube(s) in which the at least one needle is disposed. The distance between the distal end of third handle portion 420 and a proximal end of second handle portion 418 may define an extension length for the at least one needle relative to the guide tube(s) in which the at least one needle is/are disposed.

In some examples, third handle portion 420 includes a first section 428 and a second section 430. First and second sections 428 and 430 may be selectively movable relative to each other to define an extension length for the at least one needle coupled to second inner body portion 412. For example, movement of second section 430 away from first section 428 (followed by locking or otherwise restraining first and second sections 428 and 430 relative to each other) may reduce a distance that the at least one needle can be extended, while movement of second section 430 toward first section 428 (followed by locking or otherwise restraining first and second sections 428 and 430 relative to each other) may increase a distance that the at least one needle can be extended. In some examples, first and second sections 428 and 430 are threaded to each other so that relative rotation causes relative axial movement between first and second sections 428 and 430.

Fourth handle portion 422 is coupled to third inner body portion 414, which may be coupled to at least one needle of the therapeutic elements (different from the at least one needle coupled to second inner body portion 412) and configured to transfer axial forces applied to fourth handle portion 422 to the at least one needle to extend and retract the at least one needle from the guide tube(s) in which the at least one needle is disposed. The distance between the distal end of fourth handle portion 422 and a proximal end of third handle portion 420 may define an extension length for the at least one needle relative to the guide tube(s) in which the at least one needle is/are disposed.

In some examples, fourth handle portion 422 includes a first section 432 and a second section 434. First and second sections 432 and 434 may be selectively movable relative to each other to define an extension length for the at least one needle coupled to second inner body portion 412, like the other first and second handle sections described above.

In this way, handle 404 is configured to enable clinician control of extension length of at least one needle coupled to second inner body portion 412 and separate control of extension length of at least one needle coupled to third inner body portion 414. In some examples, third and fourth handle portions 420 and 422 may be uniquely identified via a visual or tactile identifier to allow the clinician to determine which handle portion controls which needle. Similarly, the distal portion of elongated shaft 406 may include one or more radiopaque markers that correlate to the identifiers on handle portions 420 and 422 to allow the clinician to visualize which needle will be controlled by which handle portion. For instance, the guide tubes may include unique radiopaque markers that allow the clinician to distinguish the guide tubes under medical imaging and allow the clinician to associate a particular handle portion with a particular guide tube and needle.

As described above, this may allow a clinician to extend at least one needle that is oriented toward a second renal artery a further distance than one or more other needles. This may facilitate delivery of the chemical agent to nerves adjacent to the second renal artery and treatment of those nerves and nerves adjacent to the first renal artery in a single treatment. A similar concept may be extended to any number of needles and any number of handle portions.

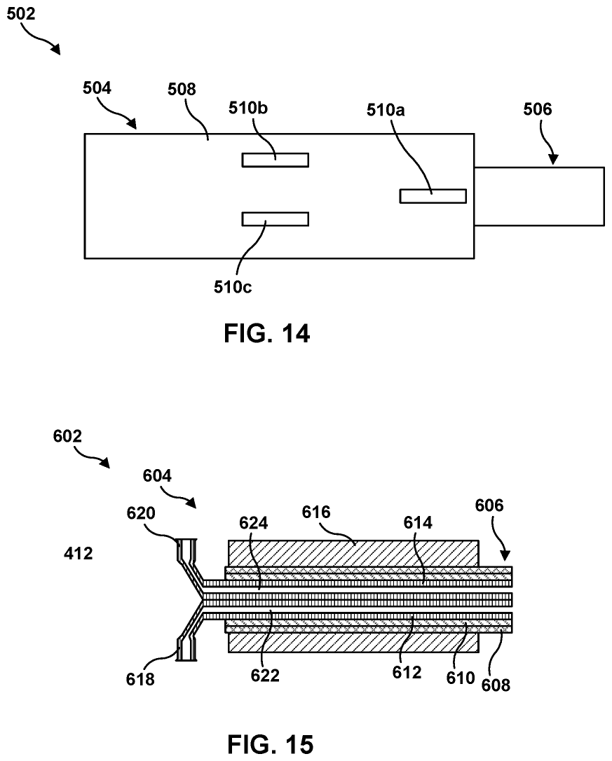

FIG. 14 is a conceptual diagram of another example neuromodulation catheter 502 that includes a handle 504 that enables allows a clinician to control the deployment distance or extension length of different needles independently. Neuromodulation catheter 502 is an example of catheters 102 and 304 of FIGS. 1 and 12, respectively. Handle 504 is coupled to or integral with elongated shaft 506, which extends to a distal end (not shown) that carries a plurality of neuromodulation elements.

Handle 504 includes a body 508 configured (e.g., shaped and sized) to be manipulated by a clinician. The clinician may apply forces to body 508 of handle 504 to transfer the forces to elongated shaft 506. For example, the clinician may apply axial forces to body 508 to distally advance or proximally withdraw elongated shaft 506 within vasculature of a patient. The clinician also may apply rotational forces about the longitudinal axis of body 508 to rotate elongated shaft 506 about its longitudinal axis, e.g., to orient therapeutic elements as described above.

Handle 504 also includes a plurality of actuators 510a-510c (“actuators 510”), three of which are shown in FIG. 14. Actuators 510 are each configured to manipulate one or more guide tube, or one or more needle to extend and retract the guide tube or needle. For instance, actuator 510 may be coupled to an inner body portion of elongated shaft 506 that is coupled to one or more guide tubes carried by the distal portion of elongated shaft 506. Clinician may operate actuator 510 to extend and retract the one or more guide tubes. For instance, first actuator 510a may be a thumbwheel coupled to a pinion gear, and a rack may be positioned within body 508 and coupled to an inner body portion of elongated shaft 506 that is coupled to the one or more guide tubes. By operating the thumbwheel in a first direction, the clinician may extend the one or more guide tubes, e.g., to place neuromodulation catheter 502 in its deployed configuration. By operating the thumbwheel in a second, opposite direction, the clinician may retract the one or more guide tubes, e.g., to return neuromodulation catheter 502 to its delivery configuration.

As another example, first actuator 510a may include a slidable tab. By sliding the slidable tab in a distal direction, the clinician may extend the one or more guide tubes. By sliding the slidable tab in a proximal direction, the clinician may retract the one or more guide tubes. First actuator 510a may take any other suitable form that enables a clinician to extend or retract the one or more guide tubes to which first actuator 510a is coupled.

Second and third actuators 510b and 510c similarly may be thumbwheels, slidable tabs, or any other suitable actuator, and the type of actuator mechanism may be independently selected for each of actuators 510. Second actuator 510b may be coupled to one or more needles and third actuator 510c may be coupled to one or more different needles. In some examples, handle 504 may include an actuator for each needle, allowing each needle to be extended and retracted independently. In other examples, two or more needles may be coupled to a single actuator and extended and retracted together.

In some examples, instead of or in addition to controlling deployment distance or extension length of at least one needle separately from one or more other needles, a neuromodulation catheter may allow control of an amount of chemical agent delivered to at least one needle separately from one or more other needles. For instance, FIG. 15 is a conceptual diagram illustrating an example neuromodulation catheter 602 that includes a proximal portion 604 that enables control of an amount of chemical agent delivered to at least one needle separately from one or more other needles. Neuromodulation catheter 602 is an example of any of the neuromodulation catheters described herein.

Proximal portion 604 of neuromodulation catheter 602 includes an elongated body 606 that includes an outer body portion 608, a first inner body portion 610, a second inner body portion 612, and a third inner body portion 614; and a handle 616. Handle 616 is coupled to outer body portion 608, similar to the example described with reference to FIG. 13. First inner body portion 610 may be coupled to one or more guide tubes at a distal portion of elongated body 606. Second and third inner body portions 612 and 614 are each coupled to one or more needles at a distal portion of elongated body 606. Like the example described with reference to FIG. 13, first, second, and third inner body portions 610, 612, and 614 enable transfer of forces from proximal portion 604 to the guide tubes and needles to extend and retract the guide tubes and needles. Although not shown in FIG. 15, proximal portion 604 may include one or more actuators (like those shown in FIG. 14) and/or neuromodulation catheter 602 may include a plurality of handles (like those shown in FIG. 13) to allow a clinician to extend and retract the guide tubes and needles collectively, individually, or combinations thereof.

In the example shown in FIG. 15, second inner body portion 612 is coupled to a first port 618 and third inner body portion 614 is coupled to a second port 620. First port 618 provides access to a lumen 622 of second inner body portion 612, and second port 620 provides access to a lumen 624 of third inner body portion 614. First and second ports 618 and 620 may be any suitable type of port that enable introduction of a chemical agent to lumens 622 and 624, respectively. For example, first and second ports 618 and 620 may include Luer connectors.

Because inner lumens of second inner body portion 612 and third inner body portion 614 are accessed using different ports 618 and 620, respectively, a clinician may use ports 618 and 620 to independently control injection of chemical agent through the needles coupled to second inner body portion 612 and third inner body portion 614. In examples in which each of second inner body portion 612 and third inner body portion 614 are connected to a single corresponding needle, this may enable independent control of the amount of chemical agent injected through each needle. In examples in which one or both of second inner body portion 612 and third inner body portion 614 are connected to two or more corresponding needles, this enables separate control of the amount of chemical agent injected through the needle(s) coupled to second inner body portion 612 and third inner body portion 614, respectively.

In some examples, instead of or in addition to including separate ports 618 and 620, a neuromodulation catheter may include one or more valves that allow a clinician to select to which needles chemical agent is being delivered. By enabling control of an amount of chemical agent delivered to individual needles or sets of needles, whether through ports and/or valves, neuromodulation catheter 602 may enable a clinician to deliver a greater amount of chemical agent to the needle oriented toward the second renal vessel. This may result in the chemical agent affecting a larger volume of tissue, which may facilitate treatment of nerves adjacent to the second renal vessel in the same treatment step as the nerves adjacent the renal vessel in which neuromodulation catheter 602 is positioned. Further, by enabling control of an amount of chemical agent delivered to individual needles or sets of needles, a clinician may more selectively target tissues known to be innervated or believed to be innervated while reducing an amount of chemical agent delivered to other tissues.

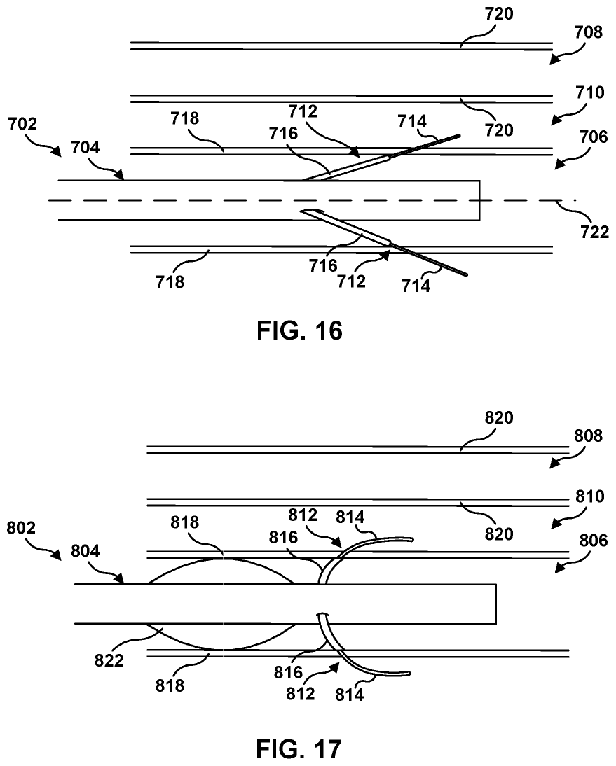

In some examples, at least part of the therapeutic elements of a neuromodulation catheter may be configured to direct chemical agent in a generally distal direction. In some patients, some renal nerves approach the renal artery nearer to the kidney than in other patients. For example, some renal nerves may not approach the renal artery until a distal aspect of the renal artery, possibly after a main bifurcation of the renal artery or even closer to the kidney. Thus, for some patients, it may be advantageous to direct chemical agent in a generally distal direction to increase a number of renal nerves that are treated. FIG. 16 is a conceptual diagram illustrating an example neuromodulation catheter 702 that includes a plurality of therapeutic elements oriented in a generally distal direction relative to neuromodulation catheter 702, which is an example of any of the neuromodulation catheters described herein.

Neuromodulation catheter 702 includes a distal portion 704 of an elongated shaft positioned within a first renal artery 706. In some instances, distal portion 704 may be positioned in a distal aspect of first renal artery 706. FIG. 16 also shows an second renal artery 708 adjacent to first renal artery 706 and separated by tissue 710. First renal artery 706 includes wall 718 and second renal artery includes wall 720. As shown in FIG. 16, neuromodulation catheter 702 includes a plurality of therapeutic elements 712 positioned circumferentially about distal portion 704.

In the example shown in FIG. 16, two therapeutic elements 712 are shown. However, neuromodulation catheter 702 may include three therapeutic elements 712, one of which is not shown because it is occluded by the body of neuromodulation catheter 702. In other examples, neuromodulation catheter 702 may include less than three therapeutic elements 712 or more than three therapeutic elements 712. Each therapeutic element 712 includes a corresponding needle 714 and, in some, but not all, examples, a corresponding guide tube 716.

Guide tubes 716 and needles 714 are illustrated in a deployed configuration. As described above with reference to FIG. 9, guide tubes 716 and needles may be movable between a delivery configuration and a deployed configuration.

In the deployed configuration, needles 714 extend generally distally with reference to neuromodulation catheter 702. Needles 714 also extend radially outward with reference to neuromodulation catheter 702. In some examples, as shown in FIG. 16, needles 714 extend in generally the same direction as guide tubes 716. This may facilitate movement of needles 714 through the corresponding guide tubes 716. In other examples, needles 714 may have a different shape from guide tubes 716 and extend in a different direction than guide tubes 716. For example, guide tubes 716 may extend more radially and less distally, while needles 714 extend more distally and less radially. In some examples, needles 714 may have pre-defined shape heat set into a shape memory alloy such as Nitinol or a spring material such as a spring steel and may adopt the pre-defined shape upon extending out of guide tubes 716.

In the example shown in FIG. 16, the plurality of needles 714 are substantially straight and define an angle with respect to a longitudinal axis 722 of distal portion 704. In some examples, the angle may be less than about 45°, or less than about 22.5°. This may enable plurality of needles 714 to direct chemical agent in a generally distal direction to increase a number of renal nerves that are treated in patients with late arriving renal nerves.

FIG. 9 (above) is another example in which needles 214 extend generally distally. In FIG. 9, needles 214 are curved, and distal portions of needles 214 extend substantially parallel to a longitudinal axis of distal portion 204. By having distal portions of needles 214 extend substantially parallel to a longitudinal axis of distal portion 204 of neuromodulation catheter 202, a possibility of adverse impact to wall 718 or other tissue due to inadvertent proximal motion of neuromodulation catheter 202 may be reduced.

In some implementations, the guide tubes (e.g., guide tubes 716 of FIG. 16) may act as a centering element configured to approximately center distal portion 704 in first renal artery 706. Guide tubes 716 also may help retain distal portion 704 in position during movement of first renal artery 706, e.g., due to patient movement, breathing, and the like. In other examples, a neuromodulation catheter may include an additional centering element.

FIG. 17 is a conceptual diagram illustrating an example neuromodulation catheter 802 that includes a plurality of therapeutic elements 812 and an expandable centering element 822. Neuromodulation catheter 802 may be similar to or substantially the same as neuromodulation catheter 202 of FIG. 9, aside from the differences described herein.

Distal portion 804 of an elongated shaft of neuromodulation catheter 802 is positioned within a first renal artery 806. In some instances, distal portion 804 may be positioned in a distal aspect of first renal artery 806. FIG. 17 also shows an second renal artery 808 adjacent to first renal artery 806 and separated by tissue 810. First renal artery 806 includes wall 818 and second renal artery 808 includes wall 820. As shown in FIG. 17, neuromodulation catheter 802 includes a plurality of therapeutic elements 812 positioned circumferentially about distal portion 804.

In the example shown in FIG. 17, two therapeutic elements 812 are shown. However, neuromodulation catheter 802 includes three therapeutic elements 812, one of which is not shown because it is occluded by the body of neuromodulation catheter 802. Again, in other examples, neuromodulation catheter 802 may include a different number of therapeutic elements 812. Each therapeutic element 812 includes a corresponding needle 814 and, optionally, a corresponding guide tube 816.

Distal portion 804 also includes expandable centering element 822. Expandable centering element may be located adjacent to therapeutic elements 812, e.g., proximally, as shown in FIG. 17, distally, or both. Expandable centering element 822 may include an inflatable balloon, an expandable wire basket, or the like. Expandable centering element 822 is configured to be carried by distal portion 804 in a relatively low-profile delivery configuration, in which expandable centering element 822 is not expanded or less expanded than a deployed configuration, and to expand to the deployed configuration after distal portion 804 is positioned within first renal artery 806. In the deployed configuration, expandable centering element 822 contacts wall 818 of first renal artery 806, approximately centers distal portion 804 within first renal artery 806 and may help retain distal portion 804 in position relative to wall 818 during the treatment. Although a single expandable centering element 822 is shown in FIG. 17, in other examples, neuromodulation catheter 802 may include multiple expandable centering elements 822, e.g., one expandable centering element 822 proximal of therapeutic elements 812 and one expandable centering element 822 distal of therapeutic elements 812.

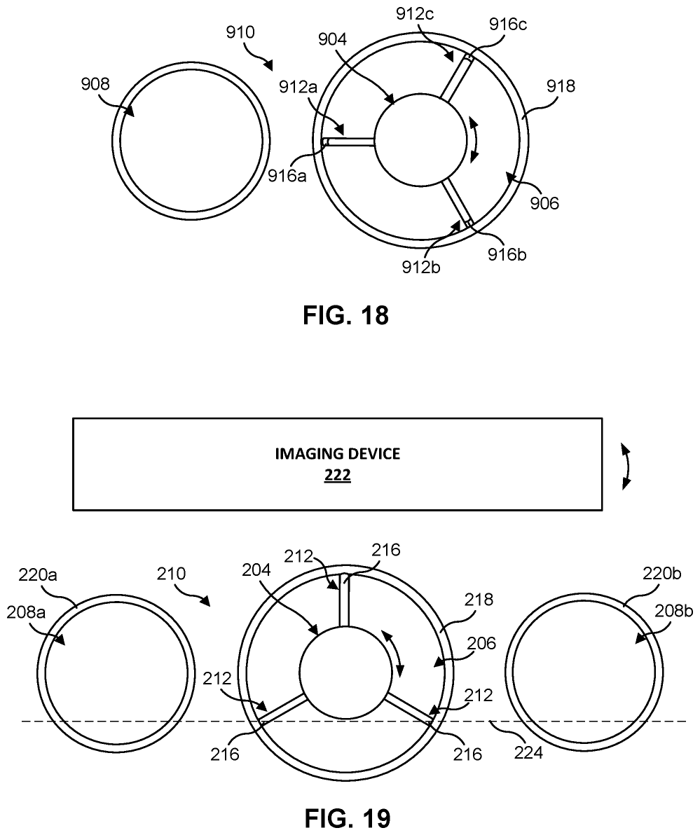

In some examples, as described above, instead of performing neuromodulation therapy using a chemical agent, techniques described herein may use perform therapy using neuromodulation energy. FIG. 18 is a conceptual diagram illustrating a schematic cross-sectional view of an example neuromodulation catheter 904 that includes a plurality of therapeutic elements 912 configured to deliver neuromodulation energy, in accordance with some examples of the present disclosure. Neuromodulation catheter 904 is positioned within a first renal vessel 906 and. A second renal vessel 908 is adjacent to first renal vessel 906, with tissue 910 between first renal vessel 906 and second renal vessel 908. As described throughout, first renal vessel 906 may be a main renal vessel and second renal vessel 908 may be an accessory renal vessel, or vice versa.

Therapeutic elements 912a-912c (“therapeutic elements 912”) each include a corresponding energy delivery element 916a-916c (“energy delivery elements 916”). Energy delivery elements 916 may include electrodes configured to deliver radiofrequency (RF) energy, microwave energy, or the like; may include ultrasound transducers configured to deliver ultrasound energy; or the like. As shown in FIG. 18, in some examples, therapeutic elements 912 may be deployed to contact an inner surface of vessel wall 918. In other examples, therapeutic elements 912 may be positioned within first renal vessel 906 and may not contact vessel wall 918.

Energy delivery to and via energy delivery elements 916 may be controlled independently, such that a selected amount of energy may be delivered to and each of energy delivery elements 916. This may allow delivery of greater energy by energy delivery element 912a, which is oriented toward second renal vessel 908 and used to deliver neuromodulation therapy to nerves adjacent to both first renal vessel and second renal vessel, and delivery of lesser energy by energy delivery elements 912b and 912c, which may be used to deliver neuromodulation therapy to nerves adjacent to first renal vessel 906 but not second renal vessel 908. Similar techniques to those described throughout this application may be used to position and orient neuromodulation catheter 904 and neuromodulation elements 912.

In some implementations, the techniques described herein may be extended to treating three renal vessels substantially simultaneously. For example, FIG. 19 is a conceptual diagram illustrating a schematic cross-sectional view of an example neuromodulation catheter 204 that includes a plurality of therapeutic elements 212 configured to deliver neuromodulation therapy to three renal vessels from a single deployment location, in accordance with some examples of the present disclosure. The components and structures shown FIG. 19 are substantially the same as those shown in FIG. 9, aside from the differences described herein.

In the example of FIG. 19, the patient includes three renal arteries 206, 208a, and 208b. Any of the three renal arteries 206, 208a, and 208b may be a main renal artery, and the others may be accessory renal arteries. As shown in FIG. 19, a first therapeutic element 212 is oriented toward second renal artery 208a and a second therapeutic element 212 is oriented toward third renal artery 208b. This may allow treatment of nerves adjacent to second a third renal arteries 208b and 208c while neuromodulation catheter 204 is positioned in first renal artery 206.

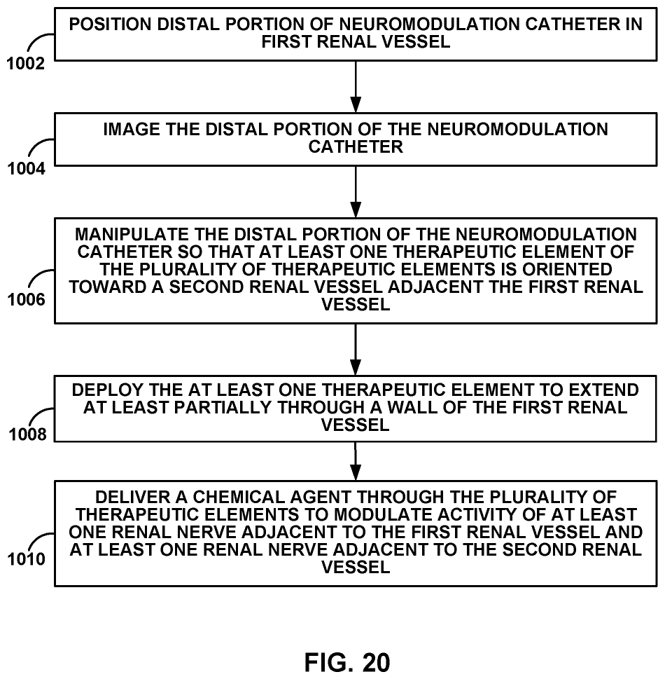

FIG. 20 is a flow diagram illustrating an example technique of performing neuromodulation of renal nerves in accordance with some examples of this disclosure. The technique of FIG. 20 will be described with concurrent reference to neuromodulation catheter 202 of FIG. 9, although it will be appreciated that the technique of FIG. 20 may be performed with other neuromodulation catheters, such as other neuromodulation catheters described herein. Conversely, it will also be appreciated that neuromodulation catheter 202 of FIG. 9 may be used in other techniques.

The technique of FIG. 20 includes positioning a distal portion 204 of a neuromodulation catheter 202 in a renal vessel of a patient (1002). A clinician may access the renal vessel of the patient through an intravascular path, such as a percutaneous access site in the femoral, brachial, radial, or axillary artery to a targeted treatment site within the renal vessel. In some examples, the renal vessel may be a main renal artery, and the main renal artery may be adjacent a second renal vessel, such as an accessory renal artery. By manipulating a proximal portion of neuromodulation catheter 202 from outside the intravascular path, a clinician may advance at least distal portion 204 through the sometimes tortuous intravascular path and remotely manipulate distal portion 204.

At least the distal portion 204 of neuromodulation catheter 202 then may be imaged (1004). For example, imaging device 222 may be used to image distal portion 204 and the surrounding physiological environment, including at least portions of first renal artery 206 and second renal artery 208. In some implementations, imaging device 222 may be manipulable to orient image plane 224 relative to distal portion 204, first renal artery 206, and second renal artery 208. For instance, in examples in which imaging device 222 is a fluoroscopic c-arm machine, the c-arm may be manipulated to control image plane 224 relative to distal portion 204, first renal artery 206, and second renal artery 208.

In some examples, imaging device 222 may be manipulated so that at least a portion of first renal artery 206 and second renal artery 208 lie substantially within image plane 224. A clinician may manipulate imaging device 222 so that imaging plane 224 is substantially parallel to the longitudinal axis of first renal artery 206 and the longitudinal axis of second renal artery 208. Further, in some implementations, a clinician may control a focal depth of imaging device 222 so that image plane 224 is within the portion of first renal artery 206 and the portion of second renal artery 208. This may facilitate placement and orientation of distal portion 204 within first renal artery 206 with at least one of therapeutic elements 212 oriented toward second renal artery 208.

The clinician then may manipulate distal portion 204 of neuromodulation catheter 202 so that at least one therapeutic element of the plurality of therapeutic elements 212 is oriented toward second renal artery 208 adjacent first renal artery 206 (1006). For example, as described above, guide tubes 216 may be radiopaque or may include a radiopaque marker or band, e.g., near a distal end of each guide tube. The clinician may at least partially deploy guide tubes 216 so that guide tubes are visible in the image and may manipulate a proximal portion or a handle of neuromodulation catheter 202 to rotate neuromodulation catheter 202 so that at least one therapeutic element of the plurality of therapeutic elements 212 is oriented toward second renal artery 208 adjacent first renal artery 206.

Once at least one therapeutic element of the plurality of therapeutic elements 212 is oriented toward second renal artery 208 adjacent first renal artery 206, the clinician may deploy the at least one therapeutic element, and possible all the plurality of therapeutic elements 212 to extend at least partially through wall 218 of first renal artery 206 (1008). This results in the at least one therapeutic element extending toward second renal artery 208. For example, the clinician may deploy the at least one therapeutic element of the plurality of therapeutic elements 212 that is oriented toward second renal artery 208 to extend fully through wall 218 while deploying one or more other therapeutic elements to extend into the adventitia or peri-adventitia of wall 218. As described above, in some examples, all therapeutic elements are deployed together and are not separately deployable. In other example, one or more therapeutic elements (e.g., one or more needles 214) may be separately deployable from one or more other therapeutic elements, e.g., using the devices and techniques described with reference to FIGS. 12-14.

Once the at least one therapeutic element, and possibly all the plurality of therapeutic elements 212 have been deployed to extend at least partially through wall 218 of first renal artery 206 (1008), the clinician may deliver a chemical agent through the plurality of therapeutic elements to modulate activity of at least one renal nerve adjacent to first renal artery 206 and at least one renal nerve adjacent to second renal artery 208 (1010). As described above, in some examples, chemical agent is delivered through needles 214 simultaneously and in the same amount. In another example, chemical agent may be delivered through one or more therapeutic elements (e.g., one or more needles 214) separately from one or more other therapeutic elements, e.g., using the devices and techniques described with reference to FIG. 15.

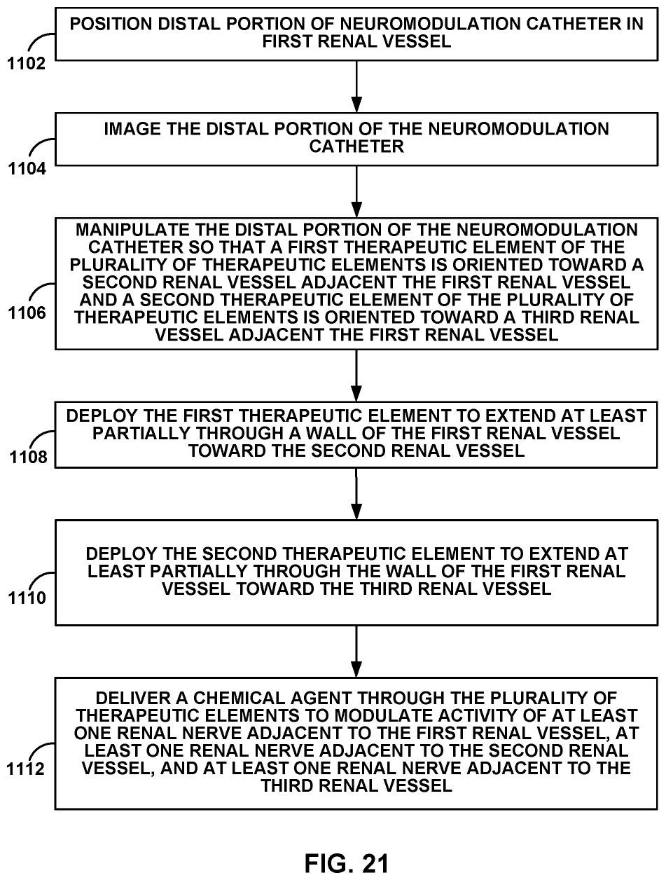

FIG. 21 is a flow diagram illustrating an example technique of performing neuromodulation of renal nerves, in accordance with some examples of this disclosure. The technique of FIG. 21 will be described with concurrent reference to neuromodulation catheter 202 of FIG. 19, although it will be appreciated that the technique of FIG. 21 may be performed with other neuromodulation catheters, such as other neuromodulation catheters described herein. Conversely, it will also be appreciated that neuromodulation catheter 202 of FIG. 19 may be used in other techniques.

The technique of FIG. 21 includes positioning a distal portion 204 of a neuromodulation catheter 202 in a first renal artery 206 of a patient (1102). This may be similar to or substantially the same as step (1002) of FIG. 20. At least the distal portion 204 of neuromodulation catheter 202 then may be imaged (1104). This may be similar to or substantially the same as step (1004) of FIG. 20.

The clinician then may manipulate distal portion 204 of neuromodulation catheter 202 so that a first therapeutic element of the plurality of therapeutic elements 212 is oriented toward a second renal artery 208a adjacent first renal artery 206 and a second therapeutic element of the plurality of therapeutic elements 212 is oriented toward a third renal artery 208b adjacent first renal artery 206 (1006). For example, as described above, guide tubes 216 may be radiopaque or may include a radiopaque marker or band, e.g., near a distal end of each guide tube. The clinician may at least partially deploy guide tubes 216 so that guide tubes are visible in the image, and may manipulate a proximal portion or a handle of neuromodulation catheter 202 to rotate neuromodulation catheter 202 so that a first therapeutic element of the plurality of therapeutic elements 212 is oriented toward a second renal artery 208a adjacent the first renal artery 206 and a second therapeutic element of the plurality of therapeutic elements 212 is oriented toward a third renal artery 208b adjacent the first renal artery 206 (1006).

The clinician then may deploy the first therapeutic element to extend at least partially through wall 218 of first renal artery 206 toward second renal artery 208a (1108) and may deploy the second therapeutic element to extend at least partially through wall 218 of first renal artery 206 toward third renal artery 208b (1110). This results in the first therapeutic element extending toward second renal artery 208a and the second therapeutic element extending toward third renal artery 208b.

Once the first and second therapeutic elements, and possibly all the plurality of therapeutic elements 212 have been deployed to extend at least partially through wall 218 of main renal artery 206 (1108) and (1110), the clinician may deliver a chemical agent through the plurality of therapeutic elements to modulate activity of at least one renal nerve adjacent to first renal artery 206, at least one renal nerve adjacent to second renal artery 208a, and at least one renal nerve adjacent to third renal artery 208b (1212). As described above, in some examples, chemical agent is delivered through needles 214 simultaneously and in the same amount. In another example, chemical agent may be delivered through one or more therapeutic elements (e.g., one or more needles 214) separately from one or more other therapeutic elements, e.g., using the devices and techniques described with reference to FIG. 15.

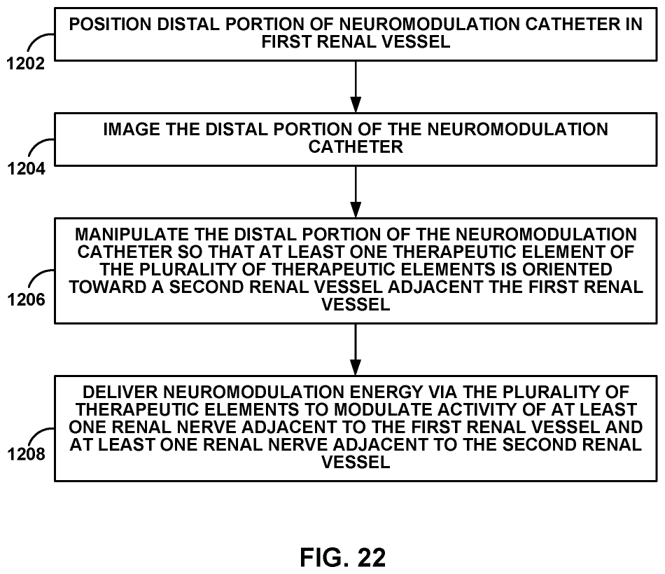

FIG. 22 is a flow diagram illustrating an example technique of performing neuromodulation of renal nerves, in accordance with some examples of this disclosure. The technique of FIG. 22 will be described with concurrent reference to the neuromodulation catheter of FIG. 18, although it will be appreciated that the technique of FIG. 22 may be performed with other neuromodulation catheters, such as other neuromodulation catheters described herein. Conversely, it will also be appreciated that neuromodulation catheter of FIG. 18 may be used in other techniques.

The technique of FIG. 22 includes positioning a distal portion 904 of a neuromodulation catheter in a first renal artery 906 of a patient (1202). This may be similar to or substantially the same as step (1002) of FIG. 20. At least the distal portion 904 of the neuromodulation catheter then may be imaged (1204). This may be similar to or substantially the same as step (1004) of FIG. 20. The clinician then may manipulate distal portion 904 of the neuromodulation catheter so that at least one therapeutic element of the plurality of therapeutic elements 912 is oriented toward second renal artery 908 adjacent first renal artery 906 (1206). This may be similar to or substantially the same as step (1006) of FIG. 20.

The clinician then may deliver neuromodulation energy via the plurality of therapeutic elements 912 to modulate activity of at least one renal nerve adjacent to first renal artery 906 and at least one renal nerve adjacent to the second renal artery 908 (1208). In some implementations, a greater amount of neuromodulation energy to be delivered via the at least one therapeutic element 912a of the plurality of therapeutic elements 912 that is oriented toward second renal artery 908 than is delivered via at least one other therapeutic element of the plurality of therapeutic elements. This may facilitate neuromodulation of nerves adjacent to second renal artery 908 in the same treatment step as nerves adjacent to first renal artery 906.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure. For example, while particular features of the neuromodulation catheters were described as being part of a single device, in other examples, these features can be included on one or more separate devices that can be positioned adjacent to and/or used in tandem with the neuromodulation catheters to perform similar functions to those described herein. Additionally, while the description of the present technology is focused on delivering chemical agents, the present technology can equally be applied to other methods of neuromodulation therapy, including cooling, heating, electrical stimulation (using needle electrodes), RF energy delivery (using needle electrodes), microwave energy delivery (using microwave needles), ultrasound (using ultrasound transducers), or the like.

Certain aspects of the present disclosure described in the context of particular examples may be combined or eliminated in other embodiments. For example, the actuators 510 of FIG. 14 the handles 416, 418, 420, and/or 422 of FIG. 13, and/or the ports 616 and 618 of FIG. 15 may be combined in any suitable manner. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within the single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique, or any combination thereof.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “about” or “approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

The scope of the present disclosure includes the following non-limiting examples.

Example 1: A method includes positioning a distal portion of a neuromodulation catheter in a first renal vessel of a patient, wherein the distal portion of the neuromodulation catheter comprises a plurality of therapeutic elements arranged around a perimeter of the distal portion of the neuromodulation catheter, and wherein, in a deployed configuration, the plurality of therapeutic elements are configured to extend at least partially through a wall of the first renal vessel; imaging the distal portion of the neuromodulation catheter to visualize positions of the plurality of therapeutic elements; manipulating the distal portion of the neuromodulation catheter so that at least one therapeutic element of the plurality of therapeutic elements is oriented toward a second renal vessel adjacent the first renal vessel; deploying the at least one therapeutic element to extend at least partially through the wall of the first renal vessel such that the at least therapeutic element extends toward the second renal vessel; and delivering a chemical agent through the plurality of therapeutic elements to modulate activity of at least one renal nerve adjacent to the first renal vessel and at least one renal nerve adjacent to the second renal vessel.

Example 2: The method of example 1, wherein positioning the distal portion of the neuromodulation catheter in renal vessel of the patient comprises positioning the distal portion of the neuromodulation catheter in a distal aspect of a main renal artery, wherein the second renal vessel is an accessory renal artery.

Example 3: The method of example 1, wherein positioning the distal portion of the neuromodulation catheter in renal vessel of the patient comprises positioning the distal portion of the neuromodulation catheter in a distal aspect of an accessory renal artery, wherein the second renal vessel is a main renal artery.

Example 4: The method of any one of examples 1 to 3, further comprising, prior to deploying the at least one therapeutic element, determining relative positions of the second renal vessel and the first renal vessel.

Example 5: The method of example 4, wherein determining the relative positions of the second renal vessel and the first renal vessel comprises imaging a physiological environment within a body of the patient that includes the second renal vessel and the first renal vessel.

Example 6: The method of example 4 or example 5, wherein determining the relative positions of the second renal vessel and the first renal vessel comprises manipulating an imaging device so that at least part of the first renal vessel adjacent the plurality of therapeutic elements and at least part of the second renal vessel adjacent the plurality of therapeutic elements are substantially in an image plane.

Example 7: The method of any one of examples 1 to 6, wherein each therapeutic element comprises a radiopaque portion.

Example 8: The method of any one of examples 1 to 7, wherein each therapeutic element is independently deployable from the neuromodulation catheter.

Example 9: The method of example 8, wherein a deployment distance of each therapeutic element is independently controllable.

Example 10: The method of any one of examples 1 to 9, wherein deploying the at least one therapeutic element comprises deploying the at least one therapeutic element to extend fully through the wall of the first renal vessel.

Example 11: The method of example 10, wherein deploying the at least one therapeutic element comprises deploying the at least one therapeutic element to extend fully through the wall of the first renal vessel and to the second renal vessel.

Example 12: The method of example 10 or example 11, further comprising deploying at least one other therapeutic element a shorter distance than the at least one therapeutic element.

Example 13: The method of any one of examples 1 to 12, wherein the chemical agent comprises an alcohol.

Example 14: The method of any one of examples 1 to 13, wherein delivering the chemical agent through the at least one therapeutic element comprises independently controlling an amount of chemical agent delivered through each therapeutic element of the plurality of therapeutic elements.

Example 15: The method of example 14, further comprising delivering more chemical agent through the at least one therapeutic element than an amount of chemical agent delivered through at least one other therapeutic element.

Example 16: The method of any one of examples 1 to 15, wherein the perimeter is a circumference, and wherein the plurality of therapeutic elements comprise three therapeutic elements, and wherein the three therapeutic elements are spaced substantially equally around the circumference of the neuromodulation catheter.

Example 17: The method of any one of examples 1 to 16, wherein each therapeutic element comprises a needle.

Example 18: The method of example 17, wherein each therapeutic element further comprises a guide tube, and wherein the needle of each therapeutic element is configured to be moved relative to the corresponding guide tube.

Example 19: The method of example 17 or example 18, wherein, when deployed, the plurality of needles are oriented in a generally distal direction with reference to the neuromodulation catheter.

Example 20: The method of any one of examples 17 to 19, wherein, when deployed, the plurality of needles substantially straight and define an angle of less than 45 degrees with respect to a longitudinal axis of the neuromodulation catheter.

Example 21: The method of example 20, wherein, when deployed, the plurality of needles substantially straight and define an angle of less than 22.5 degrees with respect to a longitudinal axis of the neuromodulation catheter.

Example 22: The method of any one of examples 17 to 21, wherein, when deployed, distal portions of the plurality of needles are in tissue outside the first renal vessel, and wherein the distal portions of the plurality of needles are substantially parallel to a longitudinal axis of the neuromodulation catheter.

Example 23: The method of any one of examples 17 to 22, wherein the plurality of needles comprise a shape memory alloy.

Example 24: The method of any one of examples 1 to 23, further comprising, after manipulating the distal portion of the neuromodulation catheter so that the at least one therapeutic element of the plurality of therapeutic elements is oriented toward the second renal vessel and before deploying the at least one therapeutic element, deploying at least one centering element to substantially center the neuromodulation catheter within the first renal vessel.

Example 25: The method of example 24, wherein each therapeutic element comprises a guide tube, and wherein deploying the at least one centering element comprising deploying the plurality of guide tubes to contact the wall of the renal vessel.

Example 26: The method of example 24 or example 25, wherein deploying the at least one centering element comprising deploying an expandable centering element.

Example 27: The method of example 26, wherein the expandable centering element comprises at least one of an inflatable balloon or an expandable wire basket.

Example 28: A method includes positioning a distal portion of a neuromodulation catheter in a first renal vessel of a patient, wherein the distal portion of the neuromodulation catheter comprises a plurality of therapeutic elements arranged around a perimeter of the distal portion of the neuromodulation catheter, and wherein, in a deployed configuration, the plurality of therapeutic elements are configured to extend at least partially through a wall of the first renal vessel; imaging the distal portion of the neuromodulation catheter to visualize positions of the plurality of therapeutic elements; manipulating the distal portion of the neuromodulation catheter so that a first therapeutic element of the plurality of therapeutic elements is oriented toward a second renal vessel adjacent the first renal vessel and a second therapeutic element of the plurality of therapeutic elements is oriented toward a third renal vessel adjacent the first renal vessel; deploying the first therapeutic element to extend at least partially through the wall of the first renal vessel such that the first therapeutic element extends toward the second renal vessel; deploying the second therapeutic element to extend at least partially through the wall of the first renal vessel such that the second therapeutic element extends toward the third renal vessel; and delivering a chemical agent through the plurality of therapeutic elements to modulate activity of at least one renal nerve adjacent to the first renal vessel, at least one renal nerve adjacent to the second renal vessel, and at least one renal nerve adjacent to the third renal vessel.

Example 29: The method of example 28, wherein the first renal vessel comprises a first renal artery extending from an aorta of the patient toward a kidney of the patient, wherein the second renal vessel comprises a second renal artery extending from the aorta of the patient toward the kidney of the patient, and wherein the third renal vessel comprises a third renal artery extending from the aorta of the patient toward the kidney of the patient, and wherein the second renal artery is on a first side of the first renal artery, and wherein the third renal artery is on a second, different side of the first renal artery.

Example 30: The method of example 29, wherein the first renal artery is a main renal artery, wherein the second renal artery is a first accessory renal artery, and wherein the third renal artery is a second accessory renal artery.

Example 31: The method of example 29, wherein the first renal artery is a first accessory renal artery, wherein the second renal artery is a main renal artery, and wherein the third renal artery is a second accessory renal artery.

Example 32: The method of any one of examples 28 to 31, further comprising, prior to deploying the first therapeutic element and deploying the second therapeutic element, determining relative positions of the first renal vessel, the second renal vessel, and the third renal vessel.

Example 33: The method of example 32, wherein determining the relative positions of the first renal vessel, the second renal vessel and the third renal vessel comprises imaging a physiological environment within a body of the patient that includes the first renal vessel, the second renal vessel, and the third renal vessel.

Example 34: The method of example 32 or example 33, wherein determining the relative positions of the first renal vessel, the second renal vessel, and the third renal vessel comprises manipulating an imaging device so that at least part of the first renal vessel adjacent the plurality of therapeutic elements, at least part of the second renal vessel adjacent the plurality of therapeutic elements, and at least part of the third renal vessel adjacent the plurality of therapeutic elements are substantially in an image plane.

Example 35: The method of any one of examples 28 to 34, wherein each therapeutic element comprises a radiopaque portion.

Example 36: The method of any one of examples 28 to 35, wherein each therapeutic element is independently deployable from the neuromodulation catheter.

Example 37: The method of example 36, wherein a deployment distance of each therapeutic element is independently controllable.

Example 38: The method of any one of examples 28 to 37, wherein deploying the first therapeutic element comprises deploying the first therapeutic element to extend fully through the wall of the first renal vessel.

Example 39: The method of example 38, further comprising deploying at least one other therapeutic element a shorter distance than the first therapeutic element.

Example 40: The method of any one of examples 28 to 39, wherein the chemical agent comprises an alcohol.

Example 41: The method of any one of examples 28 to 40, wherein delivering the chemical agent through the at least one therapeutic element comprises independently controlling an amount of chemical agent delivered through each therapeutic element of the plurality of therapeutic elements.

Example 42: The method of example 41, further comprising delivering more chemical agent through the first therapeutic element than an amount of chemical agent delivered through at least one other therapeutic element.

Example 43: The method of any one of examples 28 to 42, wherein the perimeter is a circumference, and wherein the plurality of therapeutic elements comprise three therapeutic elements, and wherein the three therapeutic elements are spaced substantially equally around the circumference of the neuromodulation catheter.

Example 44: The method of any one of examples 28 to 43, wherein each therapeutic element comprises a needle.

Example 45: The method of example 44, wherein each therapeutic element further comprises a guide tube, and wherein the needle of each therapeutic element is configured to be moved relative to the corresponding guide tube.

Example 46: The method of example 44 or example 45, wherein, when deployed, the plurality of needles are oriented in a generally distal direction with reference to the neuromodulation catheter.

Example 47: The method of any one of examples 44 to 46, wherein, when deployed, the plurality of needles substantially straight and define an angle of less than 45 degrees with respect to a longitudinal axis of the neuromodulation catheter.

Example 48: The method of any one of examples 44 to 47, wherein, when deployed, distal portions of the plurality of needles are in tissue outside the first renal vessel, and wherein the distal portions of the plurality of needles are substantially parallel to a longitudinal axis of the neuromodulation catheter.

Example 49: The method of any one of examples 44 to 48, wherein the plurality of needles comprise a shape memory alloy.

Example 50: The method of any one of examples 28 to 49, further comprising, after manipulating the distal portion of the neuromodulation catheter so that the first therapeutic element of the plurality of therapeutic elements is oriented toward the second renal vessel and before deploying the at least one therapeutic element, deploying at least one centering element to substantially center the neuromodulation catheter within the first renal vessel.

Example 51: The method of example 50, wherein each therapeutic element comprises a guide tube, and wherein deploying the at least one centering element comprising deploying the plurality of guide tubes to contact the wall of the renal vessel.

Example 52: The method of example 50 or example 51, wherein deploying the at least one centering element comprising deploying an expandable centering element.

Example 53: The method of example 52, wherein the expandable centering element comprises at least one of an inflatable balloon or an expandable wire basket.

Example 54: A method includes positioning a distal portion of a neuromodulation catheter in a first renal vessel of a patient, wherein the distal portion of the neuromodulation catheter comprises a plurality of therapeutic elements arranged around a perimeter of the distal portion of the neuromodulation catheter; imaging the distal portion of the neuromodulation catheter to visualize positions of the plurality of therapeutic elements; manipulating the distal portion of the neuromodulation catheter so that at least one therapeutic element of the plurality of therapeutic elements is oriented toward a second renal vessel adjacent the first renal vessel; and delivering neuromodulation energy via the plurality of therapeutic elements to modulate activity of at least one renal nerve adjacent to the first renal vessel and at least one renal nerve adjacent to the second renal vessel, wherein a greater amount of neuromodulation energy is delivered via the at least one therapeutic element of the plurality of therapeutic elements that is oriented toward a second renal vessel than is delivered via at least one other therapeutic element of the plurality of therapeutic elements.

Example 55: The method of example 54, wherein positioning the distal portion of the neuromodulation catheter in renal vessel of the patient comprises positioning the distal portion of the neuromodulation catheter in a distal aspect of a main renal artery, wherein the second renal vessel is an accessory renal artery.

Example 56: The method of example 54, wherein positioning the distal portion of the neuromodulation catheter in renal vessel of the patient comprises positioning the distal portion of the neuromodulation catheter in a distal aspect of an accessory renal artery, wherein the second renal vessel is a main renal artery.

Example 57: The method of any one of examples 54 to 56, further comprising, prior to deploying the at least one therapeutic element, determining relative positions of the second renal vessel and the first renal vessel.

Example 58: The method of example 57, wherein determining the relative positions of the second renal vessel and the first renal vessel comprises imaging a physiological environment within a body of the patient that includes the second renal vessel and the first renal vessel.

Example 59: The method of example 57 or example 58, wherein determining the relative positions of the second renal vessel and the first renal vessel comprises manipulating an imaging device so that at least part of the first renal vessel adjacent the plurality of therapeutic elements and at least part of the second renal vessel adjacent the plurality of therapeutic elements are substantially in an image plane.

Example 60: The method of any one of examples 54 to 59, wherein each therapeutic element comprises a radiopaque portion.

Example 61: The method of any one of examples 54 to 60, wherein an amount of neuromodulation energy delivered by each therapeutic element is independently controllable.

Example 62: The method of any one of examples 54 to 61, wherein the perimeter is a circumference, and wherein the plurality of therapeutic elements comprise three therapeutic elements, and wherein the three therapeutic elements are spaced substantially equally around the circumference of the neuromodulation catheter.

Example 63: The method of any one of examples 54 to 62, wherein each therapeutic element comprises one or more of an ultrasound transducer, a radiofrequency electrode, or a microwave electrode.

Example 64: The method of any one of examples 54 to 63, further comprising, after manipulating the distal portion of the neuromodulation catheter so that the at least one therapeutic element of the plurality of therapeutic elements is oriented toward the second renal vessel and before delivering neuromodulation energy via the plurality of therapeutic elements, deploying an expandable centering element to substantially center the neuromodulation catheter within the first renal vessel.

Example 65: The method of example 64, wherein the expandable centering element comprises at least one of an inflatable balloon or an expandable wire basket.

What is claimed is:

1. A method comprising:
positioning a distal portion of a neuromodulation catheter in a first renal vessel of a patient, wherein the distal portion of the neuromodulation catheter comprises a plurality of therapeutic elements arranged around a perimeter of the distal portion of the neuromodulation catheter, and wherein, in a deployed configuration, the plurality of therapeutic elements are configured to extend at least partially through a wall of the first renal vessel;
imaging the distal portion of the neuromodulation catheter to visualize positions of the plurality of therapeutic elements;
manipulating the distal portion of the neuromodulation catheter so that at least one therapeutic element of the plurality of therapeutic elements is oriented toward a second renal vessel adjacent the first renal vessel;
deploying the at least one therapeutic element to extend at least partially through the wall of the first renal vessel such that the at least therapeutic element extends toward the second renal vessel; and
delivering a chemical agent through the plurality of therapeutic elements to modulate activity of at least one renal nerve adjacent to the first renal vessel and at least one renal nerve adjacent to the second renal vessel.

2. The method of claim 1, wherein positioning the distal portion of the neuromodulation catheter in renal vessel of the patient comprises positioning the distal portion of the neuromodulation catheter in a distal aspect of a main renal artery, wherein the second renal vessel is an accessory renal artery, or wherein positioning the distal portion of the neuromodulation catheter in renal vessel of the patient comprises positioning the distal portion of the neuromodulation catheter in a distal aspect of an accessory renal artery, wherein the second renal vessel is a main renal artery.

3. The method of claim 1, wherein each therapeutic element is independently deployable from the neuromodulation catheter.

4. The method of claim 3, wherein a deployment distance of each therapeutic element is independently controllable.

5. The method of claim 1, wherein deploying the at least one therapeutic element comprises deploying the at least one therapeutic element to extend fully through the wall of the first renal vessel.

6. The method of claim 1, further comprising deploying at least one other therapeutic element a shorter distance than the at least one therapeutic element.

7. The method of claim 1, wherein delivering the chemical agent through the at least one therapeutic element comprises independently controlling an amount of chemical agent delivered through each therapeutic element of the plurality of therapeutic elements.

8. The method of claim 7, further comprising delivering more chemical agent through the at least one therapeutic element than an amount of chemical agent delivered through at least one other therapeutic element.

9. The method of claim 1, wherein the perimeter is a circumference, and wherein the plurality of therapeutic elements comprise three therapeutic elements, and wherein the three therapeutic elements are spaced substantially equally around the circumference of the neuromodulation catheter.

10. The method of claim 1, wherein each therapeutic element comprises a needle.

11. The method of claim 10, wherein each therapeutic element further comprises a guide tube, and wherein the needle of each therapeutic element is configured to be moved relative to the corresponding guide tube.

12. The method of claim 10, wherein, when deployed, the needles of the plurality of therapeutic elements are oriented in a generally distal direction with reference to the neuromodulation catheter.

13. The method of claim 10, wherein, when deployed, distal portions of the needles of the plurality of therapeutic elements are in tissue outside the first renal vessel, and wherein the distal portions of the needles are substantially parallel to a longitudinal axis of the neuromodulation catheter.

14. The method of claim 1, further comprising, after manipulating the distal portion of the neuromodulation catheter so that the at least one therapeutic element of the plurality of therapeutic elements is oriented toward the second renal vessel and before deploying the at least one therapeutic element, deploying at least one centering element to substantially center the neuromodulation catheter within the first renal vessel.

15. The method of claim 14, wherein deploying the at least one centering element comprising deploying an expandable centering element.

16. A method comprising:
positioning a distal portion of a neuromodulation catheter in a first renal vessel of a patient, wherein the distal portion of the neuromodulation catheter comprises a plurality of therapeutic elements arranged around a perimeter of the distal portion of the neuromodulation catheter, and wherein, in a deployed configuration, the plurality of therapeutic elements are configured to extend at least partially through a wall of the first renal vessel;
imaging the distal portion of the neuromodulation catheter to visualize positions of the plurality of therapeutic elements;
manipulating the distal portion of the neuromodulation catheter so that a first therapeutic element of the plurality of therapeutic elements is oriented toward a second renal vessel adjacent the first renal vessel and a second therapeutic element of the plurality of therapeutic elements is oriented toward a third renal vessel adjacent the first renal vessel;
deploying the first therapeutic element to extend at least partially through the wall of the first renal vessel such that the first therapeutic element extends toward the second renal vessel;
deploying the second therapeutic element to extend at least partially through the wall of the first renal vessel such that the second therapeutic element extends toward the third renal vessel; and
delivering a chemical agent through the plurality of therapeutic elements to modulate activity of at least one renal nerve adjacent to the first renal vessel, at least one renal nerve adjacent to the second renal vessel, and at least one renal nerve adjacent to the third renal vessel.

17. The method of claim 16, wherein a deployment distance of each therapeutic element is independently controllable.

18. The method of claim 16, further comprising deploying at least one other therapeutic element a shorter distance than the first therapeutic element.

19. The method of claim 16, wherein delivering the chemical agent through the plurality of therapeutic elements comprises independently controlling an amount of chemical agent delivered through each therapeutic element of the plurality of therapeutic elements.

20. The method of claim 16, wherein each therapeutic element comprises a needle and wherein, when deployed, distal portions of the needles of the plurality of therapeutic elements are in tissue outside the first renal vessel, and wherein the distal portions of the plurality of needles are substantially parallel to a longitudinal axis of the neuromodulation catheter.

21. The method of claim 16, further comprising, after manipulating the distal portion of the neuromodulation catheter so that the first therapeutic element is oriented toward the second renal vessel and before deploying the first therapeutic element, deploying at least one centering element to substantially center the neuromodulation catheter within the first renal vessel.

22. A method comprising:
positioning a distal portion of a neuromodulation catheter in a first renal vessel of a patient, wherein the distal portion of the neuromodulation catheter comprises a plurality of therapeutic elements arranged around a perimeter of the distal portion of the neuromodulation catheter;
imaging the distal portion of the neuromodulation catheter to visualize positions of the plurality of therapeutic elements;
manipulating the distal portion of the neuromodulation catheter so that at least one therapeutic element of the plurality of therapeutic elements is oriented toward a second renal vessel adjacent the first renal vessel; and
delivering neuromodulation energy via the plurality of therapeutic elements to modulate activity of at least one renal nerve adjacent to the first renal vessel and at least one renal nerve adjacent to the second renal vessel, wherein a greater amount of neuromodulation energy is delivered via the at least one therapeutic element of the plurality of therapeutic elements that is oriented toward a second renal vessel than is delivered via at least one other therapeutic element of the plurality of therapeutic elements.

23. The method of claim 22, wherein an amount of neuromodulation energy delivered by each therapeutic element is independently controllable.

24. The method of claim 22, wherein each therapeutic element comprises one or more of an ultrasound transducer, a radiofrequency electrode, or a microwave electrode.

25. The method of claim 22, further comprising, after manipulating the distal portion of the neuromodulation catheter so that the at least one therapeutic element of the plurality of therapeutic elements is oriented toward the second renal vessel and before delivering neuromodulation energy via the plurality of therapeutic elements, deploying an expandable centering element to substantially center the neuromodulation catheter within the first renal vessel.
